(19) United States
(12) Patent Application Publication
(10) Pub. No.: US 2024/0331871 A1
(43) Pub. Date: Oct. 3, 2024

(54) METHOD AND DEVICE FOR GENERATING DATA PROCESSING SEQUENCE, AND NON-TRANSITORY COMPUTER-READABLE STORAGE MEDIUM STORING COMPUTER PROGRAM

(71) Applicant: SEIKO EPSON CORPORATION, TOKYO (JP)

(72) Inventor: Hideaki KASAHARA, MATSUMOTO-SHI (JP)

(21) Appl. No.: 18/622,967

(22) Filed: Mar. 31, 2024

(30) Foreign Application Priority Data
Mar. 31, 2023 (JP) 2023-056834

(51) Int. Cl. G16H 50/30; G16B 10/00

(52) U.S. Cl. CPC G16H 50/30; G16B 10/00

(57) ABSTRACT

A method for generating a data processing sequence includes: (a) generating a population of one generation; (b) allocating one of an input layer, which is one node, a filter layer, which is another node, and a parameter, to each of genes forming an individual, with reference to a reference table establishing a correspondence between the gene, and the node and the parameter, and thus generating the data processing sequence corresponding to the individual; (c) comparing output data and target data to be a target associated with subject data, and calculating an evaluation value indicating a degree of similarity between the output data and the target data; and (d) specifying the data processing sequence that satisfies a criterion, using a plurality of evaluation values corresponding to a plurality of data processing sequences, respectively.

The present application is based on, and claims priority from JP Application Serial Number 2023-056834, filed Mar. 31, 2023, the disclosure of which is hereby incorporated by reference herein in its entirety.

### BACKGROUND

### 1. Technical Field

The present disclosure relates to a technique for generating a data processing sequence.

### 2. Related Art

According to the related art, a technique of structurally expressing an individual as a tree structure by using genetic programming is known. JP-A-2007-87055 is an example of the related-art technique. In the related-art technique, an image processing sequence formed of a plurality of image processing filters combined together in a tree structure is generated by using genetic programming.

In the technique of JP-A-2007-87055, by using genetic programming, a plurality of image processing sequences are generated and an image processing sequence that satisfies a criterion is specified. In some cases, for an image processing filter, a plurality of necessary parameters for image processing are prepared for each type of parameter. When an image processing sequence is generated, such parameters are set. In the technique of JP-A-2007-87055, a parameter is added to a gene that expresses an image input layer or an image filter that forms an individual. In this case, when generating a plurality of individuals expressing a plurality of image processing sequences, it may be difficult to easily generate an individual, paying attention to a parameter such as adding a change to a parameter of a certain image processing filter. Such a problem not only occurs in the generation of an image processing sequence to process an image but also similarly occurs in the generation of a data processing sequence to process various data such as an image or a voltage change.

### SUMMARY

According to a first aspect of the present disclosure, a method for generating a data processing sequence is provided. The data processing sequence has an input layer, a filter layer, and an output layer, as nodes. The method for generating the data processing sequence includes: (a) generating a plurality of individuals in which a plurality of genes for expressing one of a type of the input layer and the filter layer, and a parameter set for the filter layer, are sequenced, and thus generating a population of one generation; (b) allocating one of the input layer, which is one of the nodes, the filter layer, which is another one of the nodes, and the parameter, to each of the genes forming the individual, with reference to a reference table establishing a correspondence between the gene, and the node and the parameter, and thus generating the data processing sequence corresponding to the individual; (c) performing data processing of subject data for each of a plurality of the data processing sequences expressed by the population, then comparing output data outputted for each of the data processing sequences, and target data to be a target associated with the subject data, and calculating an evaluation value indicating a degree of similarity between the output data and the target data; and (d) specifying the data processing sequence that satisfies a predetermined criterion, using a plurality of the evaluation values corresponding to the plurality of the data processing sequences, respectively.

According to a second aspect of the present disclosure, a device for generating a data processing sequence is provided. The data processing sequence has an input layer, a filter layer, and an output layer, as nodes. The device for generating the data processing sequence includes: a population generating unit that generates a plurality of individuals in which a plurality of genes for expressing one of a type of the input layer and the filter layer, and a parameter set for the filter layer, are sequenced, and thus generates a population of one generation; a gene translation unit that allocates one of the input layer, which is one of the nodes, the filter layer, which is another one of the nodes, and the parameter, to each of the genes forming the individual, with reference to a reference table establishing a correspondence between the gene, and the node and the parameter, and thus generates the data processing sequence corresponding to the individual; a calculation unit that performs data processing of subject data for each of a plurality of the data processing sequences expressed by the population, then compares output data outputted for each of the data processing sequences, and target data to be a target associated with the subject data, and calculates an evaluation value indicating a degree of similarity between the output data and the target data; and a specifying unit that specifies the data processing sequence that satisfies a predetermined criterion, using a plurality of the evaluation values corresponding to the plurality of the data processing sequences, respectively.

According to a third aspect of the present disclosure, a non-transitory computer-readable storage medium storing a computer program for causing a computer to execute generation of a data processing sequence is provided. The data processing sequence has an input layer, a filter layer, and an output layer, as nodes. The computer program includes: (a) a function of generating a plurality of individuals in which a plurality of genes for expressing one of a type of the input layer and the filter layer, and a parameter set for the filter layer, are sequenced, and thus generating a population of one generation; (b) a function of allocating one of the input layer, which is one of the nodes, the filter layer, which is another one of the nodes, and the parameter, to each of the genes forming the individual, with reference to a reference table establishing a correspondence between the gene, and the node and the parameter, and thus generating the data processing sequence corresponding to the individual; (c) a function of performing data processing of subject data for each of a plurality of the data processing sequences expressed by the population, then comparing output data outputted for each of the data processing sequences, and target data to be a target associated with the subject data, and calculating an evaluation value indicating a degree of similarity between the output data and the target data; and (d) a function of specifying the data processing sequence that satisfies a predetermined criterion, using a plurality of the evaluation values corresponding to the plurality of the data processing sequences, respectively.

### BRIEF DESCRIPTION OF THE DRAWINGS

FIG. 1 explains a generating system according to an embodiment.

FIG. 2 explains a storage device.

FIG. 3 shows an example of a learning data group.

FIG. 4 explains a gene table.

FIG. 5 explains a population of one generation.

FIG. 6 shows a tree-structured data processing sequence in a generating process.

FIG. 7 shows a table-formatted data processing sequence and a tree-structured data processing sequence.

FIG. 8 explains a coupling condition table.

FIG. 9 shows an example of a type table.

FIG. 10 is a flowchart showing processing executed by a generating device.

FIG. 11 is a detailed flowchart of generation processing for the coupling condition table in step S10.

FIG. 12 is a first view illustrating the generation processing for the coupling condition table.

FIG. 13 is a second view illustrating the generation processing for the coupling condition table.

FIG. 14 shows the coupling condition table.

FIG. 15 is a detailed flowchart of generation processing for a specified data processing sequence.

FIG. 16 shows a detailed flowchart of step S308.

FIG. 17 shows a threshold table referred to in the processing of step S308.

FIG. 18 is a first view illustrating step S308.

FIG. 19 is a second view illustrating step 308.

FIG. 20 shows a table-formatted data processing sequence.

FIG. 21 explains a type-referenced mutation process in a reference individual generating process.

FIG. 22 is a flowchart of a use processing process.

### DESCRIPTION OF EMBODIMENTS

### A. Embodiment

A-1. Configuration of Generating System

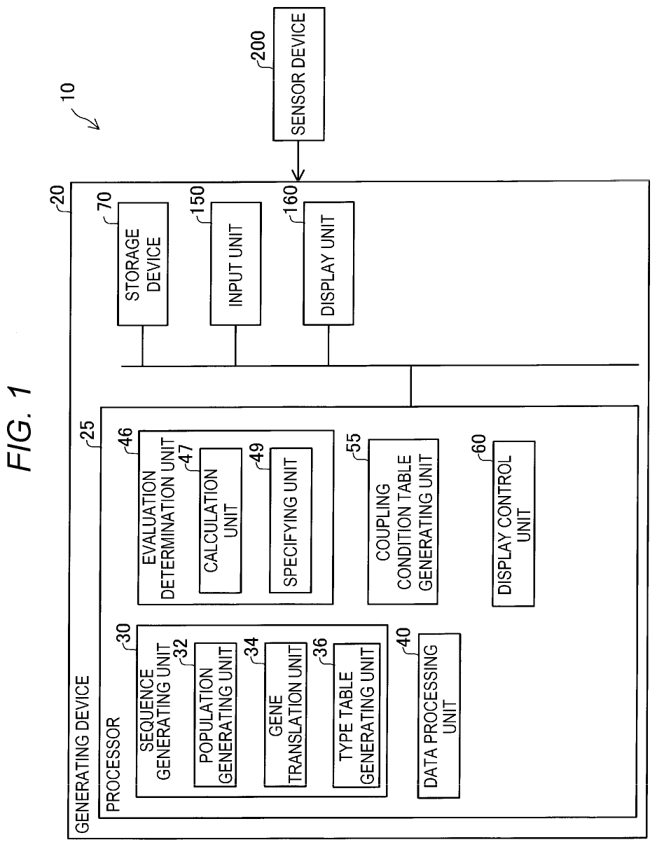

FIG. 1 explains a generating system 10 according to an embodiment. The generating system 10 has a generating device 20 and a sensor device 200. The generating device 20 and the sensor device 200 can transmit and receive data via a wire or wirelessly. The sensor device 200 is sensor that acquires a subject physical quantity. The sensor device 200 may be an image pickup device that detects an intensity of light, which is a physical quantity, and thus generates an image, or may be a voltage sensor that acquires a voltage, which is a physical quantity, or the like. In this embodiment, the sensor device 200 is an image pickup device. A picked-up image acquired by the sensor device 200 is transmitted to the generating device 20.

The generating device 20 has a sequence generating function of generating a specified data processing sequence DSq to acquire desired target data TM after data processing, a coupling condition generating function of generating a coupling condition table 88 used to execute the sequence generating function, and a data processing function. The target data TM is prepared in advance according to the purpose of data processing based on the data processing sequence. For example, when the purpose is to determine the type of input data ID inputted to a data processing sequence Sq, the target data TM is data representing the result of determination. Also, for example, when a physical quantity such as a predetermined voltage or temperature is to be acquired from the input data ID, the target data TM is data representing a physical quantity. In this embodiment, the target data TM is a target image.

The data processing function is the function of performing data processing of the input data ID, using the data processing sequence Sq. The generating device 20 in this embodiment automatically generates the specified data processing sequence DSq to acquire a desired image to achieve a desired purpose, based on a picked-up image, which is the input data ID. For example, the generating device 20 performs image processing of an image of a circuit board as an industrial product picked up by the sensor device 200, using the specified data processing sequence DSq, and thus outputs a binary image showing a defective part as a black image and the other parts as a white image, as a desired image. For example, based on this binary image, a user or a device specifies whether there is a defective part or not, and the position of a defective part, if any. Thus, an inspection of the industrial product is performed.

The sequence generating function generates a plurality of data processing sequences Sq, using genetic programming, and specifies the specified data processing sequence DSq to acquire an image after desired image processing, from among the plurality of data processing sequences Sq that are generated.

The coupling condition generating function generates the coupling condition table 88 to determine which sequence element the numeric value of a gene arranged at a gene locus corresponds to. In this embodiment, the data processing function, the sequence generating function, and the coupling condition generating function are installed in the single generating device 20. However, this is not limiting. In another embodiment, the data processing function, the sequence generating function, and the coupling condition generating function may be separately installed in different devices or may be present on a cloud.

The generating device 20 is an electronic computer such as a personal computer. The generating device 20 has a processor 25, a storage device 70, an input unit 150, and a display unit 160. The processor 25 executes various programs stored in the storage device 70 and thus controls the operation of the generating device 20. Detailed functions of the processor 25 will be described later. The storage device 70 is formed of a memory such as a RAM or a ROM. In the storage device 70, various programs to implement various functions of the processor 25, and various data used to generate the data processing sequence Sq, are stored. Details of the storage device 70 will be described later. The input unit 150 is an interface that accepts information from outside. The input unit 150, for example, accepts an input of data of a picked-up image or the like from the sensor device 200 or accepts an input of data of an image or the like generated by the user using another device. The display unit 160 displays various information. The display unit 160 is, for example, a liquid crystal monitor.

The processor 25 executes various programs in the storage device 70 and thus functions as a sequence generating unit 30, a data processing unit 40, an evaluation determination unit 46, a coupling condition table generating unit 55, and a display control unit 60. A part of the functions executed by the processor 25 may be implemented by a hardware circuit. In the present disclosure, the “processor” is a term including a CPU and a GPU.

The sequence generating unit 30 structurally expresses a gene sequence represented by an individual IV like a tree structure and thus generates a sequence set formed of a plurality of data processing sequences Sq having different combinations of sequence elements. The data processing sequence Sq is expressed by a combination of a plurality of sequence elements. The sequence elements include: (i) an input layer, which is a node to input data; (ii) a filter layer to execute data processing, which is an intermediate node between an input layer and an output layer; (iii) an output layer, which is a node to output output data PM, which is data after final data processing; and (iv) a parameter of the data processing executed by the filter layer. The data processing sequence Sq includes at least one filter layer. The input layer is a starting-point node of the data processing sequence Sq. The output layer is an end-point node of the data processing sequence Sq.

The sequence generating unit 30 has a population generating unit 32, a gene translation unit 34, and a type table generating unit 36. The population generating unit 32 generates the individual IV formed of a plurality of genes that are sequenced one-dimensionally. Each gene forming the individual IV is a numeric value representing one of the type of the input layer and the filter layer, and the parameter value of each parameter of the filter layer, of the sequence elements. One of the input layer and the filter layer, or the parameter value of the parameter, is allocated for each numeric range of the gene that represents the range of occurrence. In this way, in this embodiment, the parameter set for the filter layer, too, is expressed by a gene. As described above, the individual IV is generated by sequencing genes corresponding to the sequence elements, respectively, except the output layer.

The population generating unit 32 generates a predetermined number of individuals IV for each one generation G. In this embodiment, the population generating unit 32 sequences genes one-dimensionally and thus generates the individual IV with a predetermined gene length. In each one generation G, the combination of sequence elements varies among the plurality of data processing sequences Sq. For the first generation G, the population generating unit 32 randomly arranges a numeric value up to the third place after the decimal point, of values from 0 to 1, as a gene at each gene locus of the individual IV, and thus generates a population 84 formed of a plurality of individuals IV. From the second generation G onward, the population generating unit 32 newly generates the individual IV, using at least one of the techniques of crossover and mutation from the individual IV with a high evaluation value EV, described later, of the individuals IV of the previous parent generation G, and thus generates the population 84 that is an aggregate of the newly generated individual IV and the individual IV maintained from the parent generation G, that is, the individual IV copied from the individual IV of the parent generation G.

The gene translation unit 34 refers to a gene table 82 and the coupling condition table 88, described later, which are stored in the storage device 70, and thus translates each gene of the individual IV and generates the data processing sequence Sq expressed as a tree structure. The gene table 82 and the coupling condition table 88 are tables prescribing sequence elements corresponding to each numeric range of a numeric value, which is a gene, and the coupling relationship between the sequence elements. The data processing sequence Sq is expressed by the individual IV and the gene table 82 or by the individual IV and the coupling condition table 88.

The type table generating unit 36 executes a type table generating process of generating a type table 92 that prescribes the type of a sequence element represented by each gene of the individual IV, for each gene. The type table 92 is used to generate the individual IV generated to specify the specified data processing sequence DSq by the sequence generating function. The type table 92 is a table that prescribes a decoding type, which is the type of a sequence element represented by each gene of the individual IV, for each gene. The type table 92 prescribes the decoding type in association with all the gene locus numbers that the individual IV has, in this embodiment, 15 gene locus numbers. In this way, the type table generating process generates the type table 92 where the decoding type for distinguishing the node represented by the gene and the parameter is associated with each gene forming the individual IV from which the data processing sequence Sq is generated. Details of the type table 92 will be described later.

The data processing unit 40 performs data processing of the input data ID inputted to the input layer and outputs the output data PM after data processing from the output layer, using the data processing sequence Sq generated by the gene translation unit 34 and the specified data processing sequence DSq stored in the storage device 70.

The evaluation determination unit 46 specifies the specified data processing sequence DSq that satisfies a predetermined criterion, from among a plurality of data processing sequences Sq represented by a plurality of individuals IV of one generation G. The evaluation determination unit 46 has a calculation unit 47 and a specifying unit 49.

The calculation unit 47 compares the output data PM after data processing acquired by data processing of learning data LM, which is an example of the input data ID, based on the data processing sequence Sq represented by the individual IV, with the target data TM, which is a target associated with the learning data LM, and thus calculates the evaluation value EV indicating a degree of similarity SD between the output data PM and the target data TM. The calculation unit 47 calculates the evaluation value EV for each of a plurality of data processing sequences Sq represented by a plurality of individuals IV of one generation G. The calculated evaluation value EV is stored in association with the individual IV in the storage device 70.

The specifying unit 49 determines whether the data processing sequence Sq satisfies a predetermined criterion or not, using the calculated evaluation value EV, and specifies the data processing sequence Sq that satisfies the criterion, as the specified data processing sequence DSq.

As the evaluation value EV, for example, one of the mean squared error (MSE), the peak signal-to-noise ratio (PSNR), and the structural similarity (SSIM) can be used. When the MSE is used as the evaluation value EV, a smaller MSE value means a higher degree of similarity SD between the output data PM and the target data TM. That is, the reciprocal of the MSE represents the degree of similarity SD. When the PSNR is used as the evaluation value EV, a greater PSNR value means a higher degree of similarity SD between the output data PM and the target data TM. That is, the PSNR represents the degree of similarity SD. When the SSIM is used, for example, data such as image data is divided into sub-areas and the MSSIM (mean SSIM), which is the average value of the SSIMs calculated for the sub-areas, respectively, is used as the evaluation value EV. When the MSSIM is used as the evaluation value EV, a greater MSSIM value means a higher degree of similarity SD. That is, the MSSIM represents the degree of similarity SD. With respect to the evaluation value EV, when the output data PM and the target data TM indicate a result of determination such as whether the input data ID is a good product or a defective product, the degree of similarity SD may be, for example, the rate of coincidence between the output data PM and the target data TM corresponding to a plurality of input data ID.

The coupling condition table generating unit 55 generates the coupling condition table 88, using the evaluation value EV calculated by the evaluation determination unit 46 and the individual IV associated with the evaluation value EV. The coupling condition table 88 is a table that prescribes a coupling reference value RV indicating the suitability of the coupling of sequence elements to output desired data, using the target data TM as a target. Specifically, the coupling condition table 88 is a table that prescribes the coupling reference value RV for each element set, which is a pair of node elements made up of an input-side node element and an output-side node element coupled together. The coupling reference value RV represents the probability of occurrence of an element set of a pair of node elements as component elements of the data processing sequence Sq. The greater coupling reference value RV means a higher probability of occurrence of a corresponding element set. A method for generating the coupling condition table 88 will be described later.

The display control unit 60 causes the display unit 160 to display various information. The display control unit 60 causes the display unit 160 to display, for example, the specified data processing sequence DSq, the output data PM, the target data TM, and the evaluation value EV.

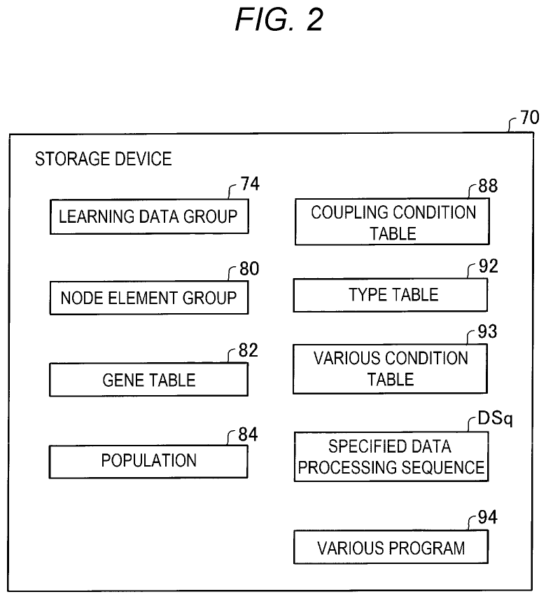

FIG. 2 explains the storage device 70. The storage device 70 stores a learning data group 74, a node element group 80, the gene table 82, the population 84, the coupling condition table 88, the type table 92, a various condition table 93, the specified data processing sequence DSq, and a various program 94.

The various condition table 93 prescribes conditions for various processing to be executed by the generating device 20. For example, the various condition table 93 prescribes an end condition used when executing the generation processing for the coupling condition table 88, a reference condition to specify the specified data processing sequence DSq, and a generation condition used when generating a population of a next generation. The specified data processing sequence DSq is the data processing sequence Sq determined as satisfying the predetermined criterion by the specifying unit 49. The specified data processing sequence DSq is represented, for example, by layer identifiers that identify a plurality of filter layers, which are a plurality of data processing layers, and the order of the layer identifiers, that is, the coupling relationship. The various program 94 is a program executed by the processor 25.

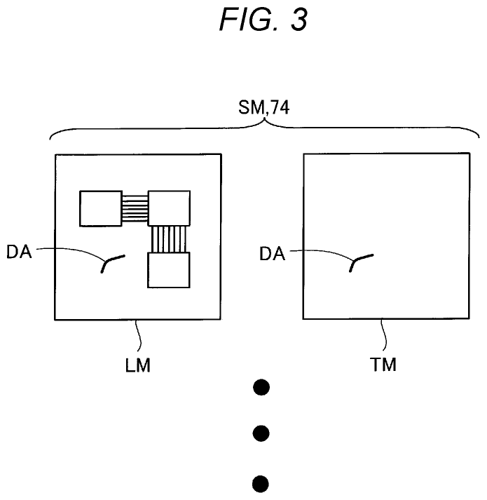

FIG. 3 shows an example of the learning data group 74. The learning data group 74 has a plurality of learning sets SM, each learning set SM being formed of the learning data LM that is the subject of the data processing based on the data processing sequence Sq, and the target data TM associated with the learning data LM. The learning data LM is, for example, an image of a circuit board, which is an industrial product. The target data TM is a binary image showing a defective part DA as a black image and the other parts as a white image. The target data TM includes two types, that is, good-product data, which is an image showing a good product, and defective-product data, which is an image showing a defective product. The defective-product data is generated, for example, by the user setting the defective part DA to be a black image and setting the other parts to be a white image, with reference to the learning data LM where the site of the defective part is specified in advance. The good-product data includes no defective part DA and is therefore set, for example, in its entirety to be a white image. The plurality of learning data LM are images of circuit boards of the same type with different serial numbers, picked up by the sensor device 200.

The node element group 80 shown in FIG. 2 is an aggregate of node elements that form nodes in the data processing sequence Sq. The node elements are a filter layer and an input layer. The filter layer may be an average filter, a maximum filter, a minimum filter, a binary filter, a moving average filter, a Laplacian filter, a Gaussian filter, a Sobel filter, a gamma correction filter, a filter that combines two data into one data, or the like. As for the filter layer, even in the case of filter layers of the same type, when a parameter value such as kernel size or coefficient can be selected, the data processing unit 40 executes data processing with a filter layer for which a selected parameter value is set.

The gene table 82 is a table showing the coupling relationship between candidate sequence elements of the data processing sequence Sq and the correspondence to sequence elements corresponding to numeric values expressed by genes.

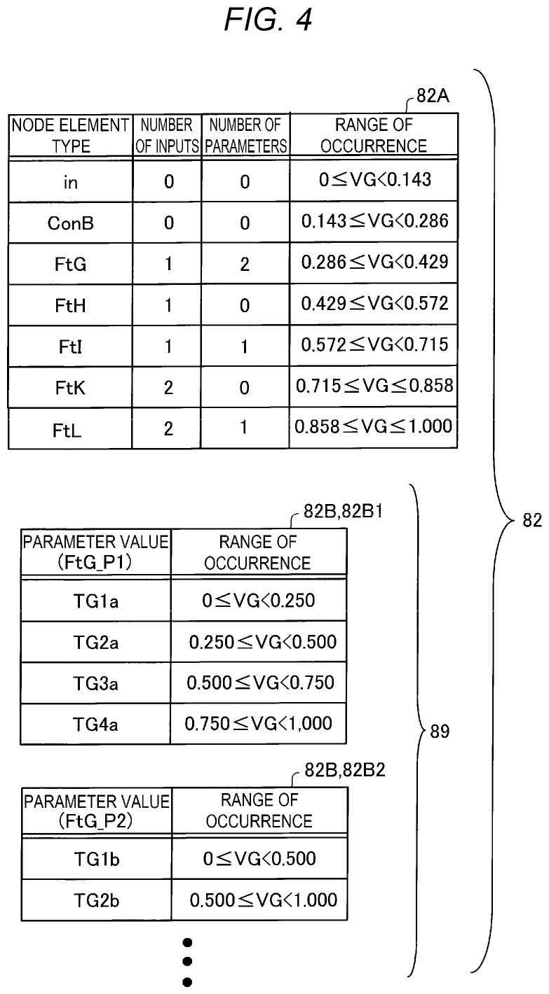

FIG. 4 explains the gene table 82. In the description below, to facilitate understanding, it is assumed that the node element group 80 is formed of five filter layers FtG to FtL. In practice, more than five filter layers are stored in the node element group 80. The gene table 82 includes a first-type gene table 82A and a second-type gene table 82B. The first-type gene table 82A shows the correspondence between input layers in, ConB and the filter layers FtG to FtL, which are node elements forming nodes in the data processing sequence Sq expressed as a tree structure, and a numeric value VG represented by a gene. The input layer in is a layer that accepts the input data ID and outputs the input data ID to the output side. The input layer ConB is a layer to which predetermined fixed data is inputted, for example, data of the same type as the input data ID. When the input data ID is an image, the fixed data is an image.

The second-type gene table 82B shows the correspondence between a parameter value and the numeric value VG expressed by a gene, when a parameter is set for the filter layer. The second-type gene table 82B is provided for each filter layer for which a parameter can be set. For example, for the filter layer FtG, two types of parameters FtG_P1, FtG_P2 can be set. Therefore, a second-type gene table 82B1 prescribes one parameter FtG_P1 and a second-type gene table 82B2 prescribes the other parameter FtG_P2.

The first-type gene table 82A prescribes the number of inputs, the number of parameters, and the range of occurrence, for each node element type representing the type of the input layer and the filter layer. The node element type is an identifier that identifies the input layer and the filter layer. The number of inputs represents the number of sequence elements coupled to the input side of the filter layer and the input layer. When the number of inputs is “2”, data outputted from two nodes are inputted to the filter layer. In this embodiment, the numbers of nodes coupled to the output side of each node are all “1”, and the filter layer and the input layer are coupled to one output-side node.

The second-type gene table 82B prescribes the relationship between the parameter value and the range of occurrence, for each node element type for which the parameter is set.

The range of occurrence in the first-type gene table 82A and the second-type gene table 82B prescribes the range of the numeric value VG of a gene. For example, in the first-type gene table 82A, when the numeric value VG of a gene is equal to or greater than 0 and smaller than 0.143, the input layer in is allocated to this gene. In this embodiment, in the gene tables 82A, 82B1, 82B2 and so on, the numeric values of 0 to 1 are equally divided to set ranges of occurrence in each table so that the node element type or the parameter value randomly occurs in the data processing sequence Sq with an equal probability.

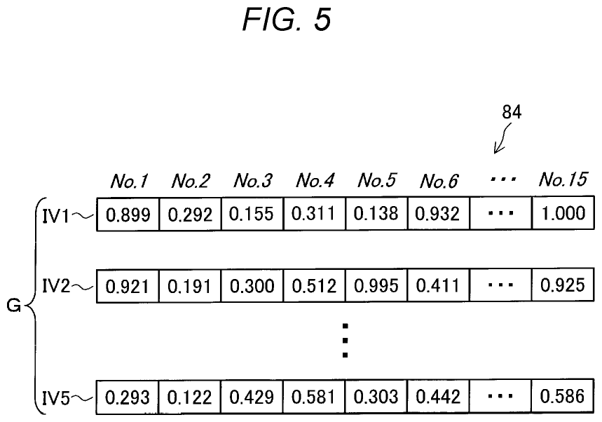

The population 84 shown in FIG. 2 stores a plurality of individuals IV of one generation G generated by the population generating unit 32. FIG. 5 explains the population 84 of one generation G. In this embodiment, an example where five individuals IV1 to IV5 are generated for one generation G is described. In each of the individuals IV1 to IV5, a plurality of gene loci where genes are arranged are sequenced one-dimensionally. The individuals IV1 to IV5 have the same gene length. Each of the individuals IV1 to IV5 is formed by sequencing genes in order at gene loci of No.1 to No. 15. The population generating unit 32 randomly arranges a gene expressed by a numeric value up to the third place after the decimal point, of values from 0 to 1, at each gene locus. That is, in this embodiment, the value that a gene can have at each gene locus is a numeric value up to the third place after the decimal point that is equal to or greater than 0 and equal to or smaller than 1.

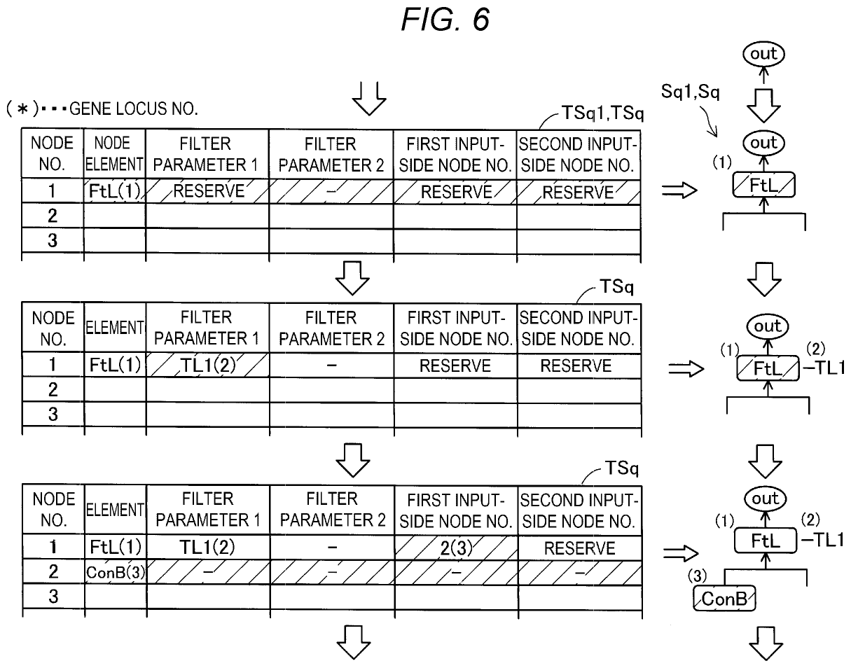

Before a further description referring to FIG. 2 is given, the generation processing for the data processing sequence Sq by the gene translation unit 34 will be described, using FIG. 6. FIG. 6 shows a tree-structured data processing sequence Sq in the generating process, generated by the gene translation unit 34. A table-formatted data processing sequence TSq representing the tree-structured data processing sequence Sq is shown on the left side in FIG. 6. The tree-structured data processing sequence Sq is shown on the right side in FIG. 6. The data processing sequence Sq is a data processing sequence Sq1 generated based on the individual IV1 shown in FIG. 5.

In this embodiment, the generation processing for the data processing sequence Sq by the gene translation unit 34 includes two types, that is, prior generation processing and specified generation processing. The prior generation processing is processing executed to execute the coupling condition generating function and thus generate the coupling condition table 88. The specified generation processing is processing executed when the sequence generating function using the coupling condition table 88 is executed. The gene translation unit 34 of the sequence generating unit 30 generates the data processing sequence Sq, referring to the gene table 82 shown in FIG. 4 in the prior generation processing, and referring to the coupling condition table 88, the second-type gene table 82B of the gene table 82, and the type table 92 in the specified generation processing. Since the prior generation processing and the specified generation processing use the same algorithm to generate the data processing sequence Sq from the individual IV, the generation of the data processing sequence Sq in the prior generation processing will be described first below.

In the prior generation processing, the gene translation unit 34 generates the tree-structured data processing sequence Sq in order from the side of the output layer out, using the individual IV, the gene table 82, and the node element group 80. As shown on the right side in FIG. 6, the gene translation unit 34 first sets the output layer out as the end-point node, which is the end of the output side, and couples a node toward the input side in the order of the sequence of the genes of the individual IV, and thus generates the data processing sequence Sq. The coupling of the sequence elements is executed according to a predetermined coupling rule. In this embodiment, the translation of the genes is executed according to a rule giving priority to depth. To describe this more in detail, the data processing sequence Sq is generated according a rule to the effect that a sequence element that is a node element or a parameter is set in the order of the sequence of the genes, giving priority to the end-point node side and that a sequence element is set, giving priority to a left-side coupling point over a right-side coupling point in a tree structure where the output layer out is at the top. That is, the filter layer or the input layer is arranged from the side of the output layer out in the order of the sequence of the genes, and when the input side is split into a plurality of parts, the left side is given priority until the sequence on the left side becomes the input layer. Also, when a parameter needs to be set for the filter layer, the next node is set after the parameter is set.

The gene translation unit 34 refers to the numeric value VG “0.899” of the gene of No.1 of the individual IV1, and the first-type gene table 82A shown in FIG. 4 to set a node, and thus specifies that “0.899” is within the range of occurrence of the filter layer FtL. Thus, the filter layer FtL is coupled to the input side of the output layer out. That is, the gene translation unit 34 sets the filter layer FtL as the node element corresponding to node No.1 in the table-formatted data processing sequence TSq. In the table-formatted data processing sequence TSq, a number in parentheses indicates a gene locus number of the individual IV1 in order to facilitate understanding.

Next, referring to the first-type gene table 82A, the gene translation unit 34 sets a reservation code in a table-formatted data processing sequence TSq1 according to the number of inputs and the number of parameters of the filter layer FtL set for node No. 1. Specifically, the number of parameters of the filter layer FtL set for node No.1 is “1” and the number of inputs is “2”. Therefore, the gene translation unit 34 sets a reservation code for a filter parameter 1, a first input-side node number, and a second input-side node number corresponding to node No.1 in the table-formatted data processing sequence TSq.

Next, the gene translation unit 34 sets a parameter for the reservation code set for the filter parameter, of the set reservation codes. Specifically, the gene translation unit 34 selects a parameter value used for the filter layer FtL, referring to the second-type gene table 82B corresponding to the filter layer FtL of node No.1, and a numeric value VG2 of the gene of gene locus No. 2. By referring to the numeric value VG2 of the gene of gene locus No.2 and the second-type gene table 82B, the gene translation unit 34 sets a parameter value TL1 for the filter layer FtL, as shown in the middle part of FIG. 6.

For the filter layer FtL set for node No.1, the gene translation unit 34 sets the input-side node after finishing the setting of the parameter. Specifically, the gene translation unit 34 sets a node, giving priority to the first input-side node, of the first input-side node and the second input-side node, until the first input-side node becomes the input layer in or the input layer ConB, which is the starting-point node. In the example shown in FIG. 6, node No. 2 is allocated to the first input-side node number of the filter layer FtL of node No.1. The gene translation unit 34 then determines the node of node No.2, referring to the numeric value of the gene of gene locus No. 3 and the first-type gene table 82A. Specifically, the numeric value of the gene of gene locus No.3 of the individual IV1 is “0.155”. Therefore, the input layer ConB whose numeric value “0.155” is within the range of occurrence is set for node No.2. That is, the input layer ConB is coupled to one input side of the two input sides of the filter layer FtL.

As the input layer ConB, which is the starting-point node, is set for the first input-side node of the filter layer FtL, the gene translation unit 34 then sets the second input-side node, for which the reservation code of node No.1 is set. The gene translation unit 34 sets a sequence element, referring to the numeric value VG of a gene at a gene locus and the gene table 82, until all the input sides become the input layer in or the input layer ConB.

When all the input-side nodes have become one of the input layer in and the input layer ConB, the gene translation unit 34 ends the generation processing for the data processing sequence Sq using the prior generation processing, even if not all the genes of the individual IV1 are used. In the case where all the genes of the individual IV1 are used, when not all the input-side nodes have become the input layer in or the input layer ConB, the gene translation unit 34 sets a predetermined input layer, in this embodiment, the input layer in, for the input-side node that is left yet to be coupled, and thus ends the generation processing for the data processing sequence Sq.

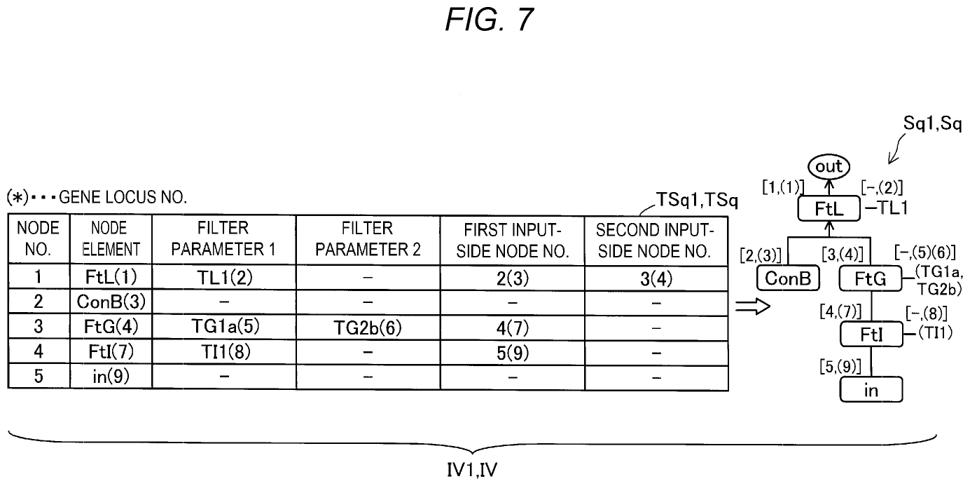

FIG. 7 shows the table-formatted data processing sequence TSq1 represented by the individual IV1 shown in FIG. 5, and a tree-structured data processing sequence Sq1. In the table-formatted data processing sequence TSq1, a number in parentheses is the corresponding gene locus number. In the tree-structured data processing sequence Sq1, the number x of [x, y] is a node number in the data processing sequence Sq, and y is the corresponding gene locus number. For the filter layer FtG, whose number of parameters is 2, a parameter TGla corresponding to gene locus No. 5 and a parameter TG2b corresponding to gene locus No. 6 are set.

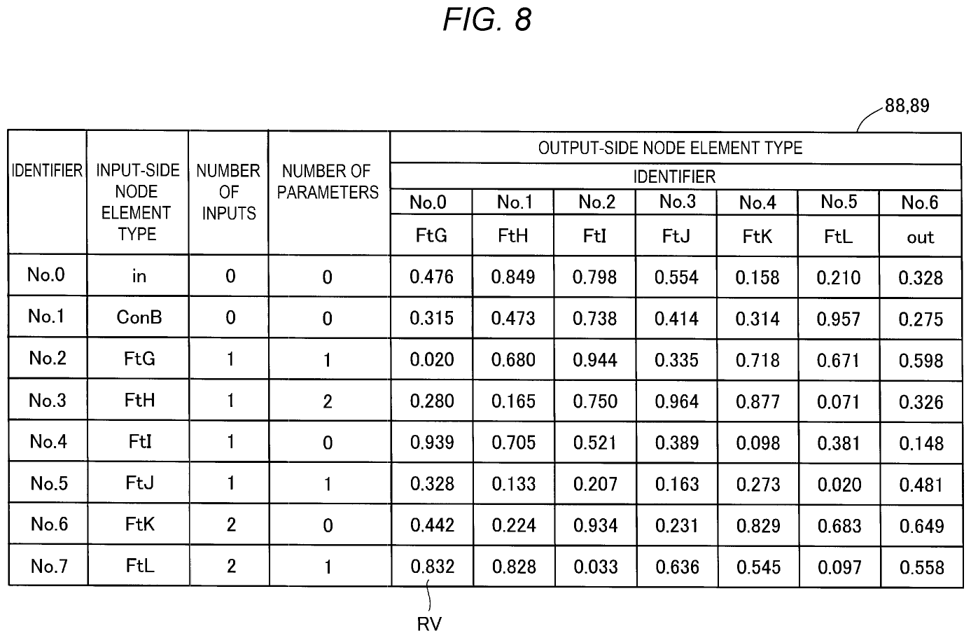

The storage device 70 will be described further, using FIG. 2. The coupling condition table 88 shown in FIG. 2 is referred to when the gene translation unit 34 generates the data processing sequence Sq from the individual IV by the specified generation processing. FIG. 8 explains the coupling condition table 88. The coupling condition table 88 prescribes an input-side node element type indicating the type of an input-side node element that is a candidate to be arranged on the input side, an identifier of the input-side node element type, an output-side node element type indicating the type of an output-side node element that is a candidate to be coupled to the input-side node, an identifier of the output-side node element type, the number of inputs, the number of parameters for each input-side node element type, and the coupling reference value RV. The input-side node elements are the input layers in, ConB and the filter layers FtG to FtL. The output-side node elements are the filter layers FtG to FtL and the output layer out. That is, the coupling condition table 88 prescribes an element set of a pair of elements made up of an input-side node element and an output-side node element coupled together. An average value ARV as the coupling reference value RV is a value prescribed for each element set and indicates the suitability of coupling in order to perform data processing of the input data ID and output a desired image, using the target data TM as a target. For example, the average value ARV of an element set where the filter layer FtG is coupled to the input side and where the filter layer FtH is coupled to the output side is “0.680”. The average value ARV is determined by the coupling condition generating function. A specific method for generating the coupling condition table 88 will be described later.

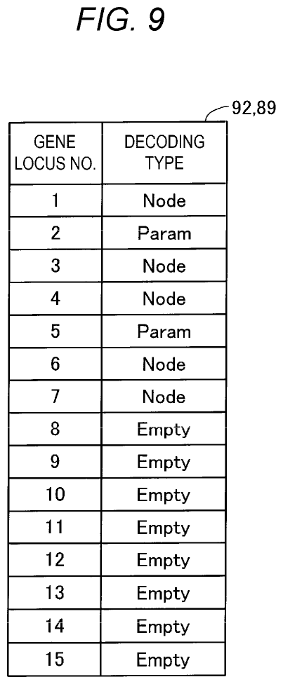

FIG. 9 shows an example of the type table 92. The type table 92 shown in FIG. 9 is generated by the type table generating unit 36 in the process of generating the data processing sequence Sq that is a candidate for the specified data processing sequence DSq by the data processing function. As shown in FIG. 9, the type table 92 is a table where a decoding type for distinguishing a node element represented by a gene and a parameter is associated with each gene arranged for the corresponding gene locus number, with respect to each individual IV. The decoding type in the type table 92 includes a type for distinguishing an unused gene that is not used for the generation of the data processing sequence Sq, in addition to distinguishing the node and the parameter. In FIG. 9, when a gene is used to represent a node element, an identifier “Node” is set as the decoding type. When a gene is used to represent a parameter, an identifier “Param” is set as the decoding type. When a gene is unused, an identifier “Empty” is set as the decoding type. The type table 92 is a part of a reference table 89.

### A-2. Processing Executed by Generating Device

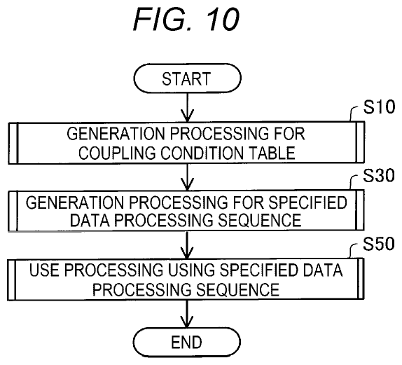

FIG. 10 is a flowchart showing processing executed by the generating device 20. First, in step S10, the coupling condition generating function of the generating device 20 executes the generation processing for the coupling condition table 88. Next, in step S30, the sequence generating function of the generating device 20 executes the generation processing for the specified data processing sequence DSq using the coupling condition table 88. Next, in step S50, the data processing function of the generating device 20 executes use processing using the specified data processing sequence DSq.

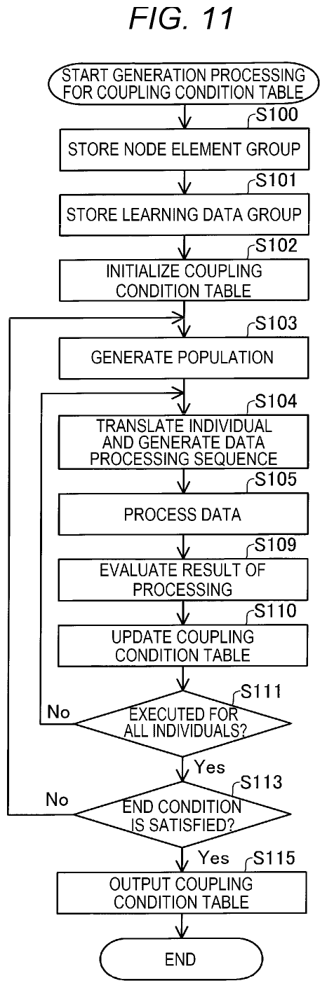

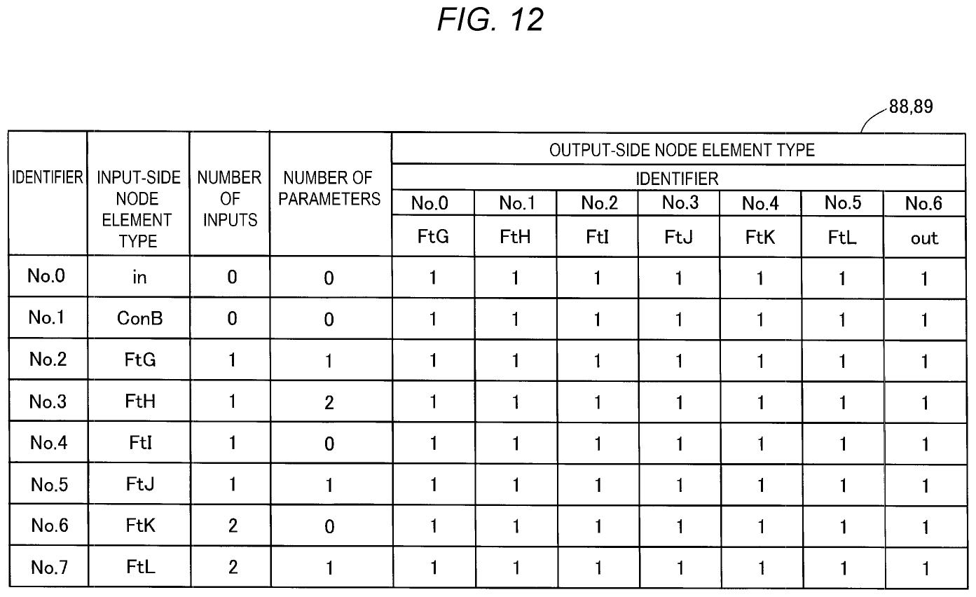

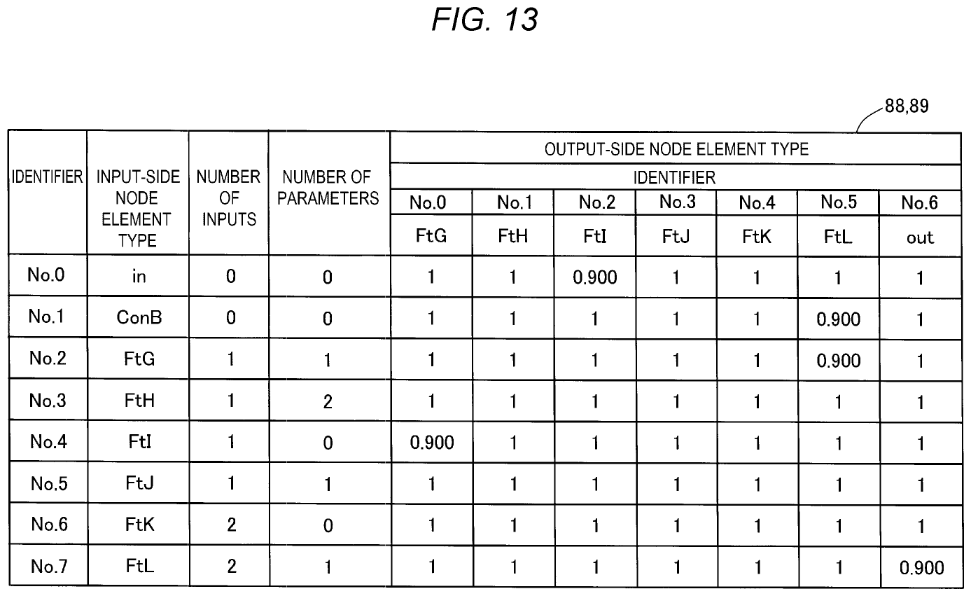

FIG. 11 is a detailed flowchart of the generation processing for the coupling condition table 88 in step S10. FIG. 12 is a first view illustrating the generation processing for the coupling condition table 88. FIG. 13 is a second view illustrating the generation processing for the coupling condition table 88.

As shown in FIG. 11, first, in step S100, the storage device 70 stores a plurality of filter layers and input layers as the node element group 80. Step S100 is executed by the user inputting, to the generating device 20, a node element such as a filter layer that is a candidate to be used in the specified data processing sequence DSq.

In step S101, the storage device 70 stores the learning data group 74 formed of a plurality of learning sets SM. Step S101 is executed by the user inputting the learning data group 74 to the generating device 20.

In step S102, the coupling condition table generating unit 55 initializes the coupling condition table 88. Specifically, the coupling condition table generating unit 55 sets all the average values ARV, which are the values of individual element sets in the coupling condition table 88, to be predetermined value, as shown in FIG. 12. As the predetermined value, any value of 0 to 1, which is the range of values that the average value ARV can be, may be set. In this embodiment, the predetermined value is “1”. The order of steps S100, S101, and S102 is not limited to this order.

Next, as shown in FIG. 11, in step S103, the population generating unit 32 generates the population 84 formed of a plurality of individuals IV of the current generation G. The population 84 of one generation G is formed of a plurality of individuals IV1 to IV5. The generated population 84 is stored in the storage device 70. Next, in step S104, the gene translation unit 34 selects one of the individuals IV1 to IV5, translates the selected one individual IV by referring to the gene table 82, and thus generates the data processing sequence Sq. The data processing sequence Sq may be stored in the storage device 70, for example, as the table-formatted data processing sequence TSq shown in FIG. 7. To “translate” means to convert each gene of the individual IV to a sequence element and thus generate the data processing sequence Sq from the individual IV.

Next, as shown in FIG. 11, in step S105, the data processing unit 40 performs data processing of a plurality of learning data LM, using the data processing sequence Sq generated in step S104, and thus generates a plurality of output data PM.

Next, in step S109, the calculation unit 47 of the evaluation determination unit 46 compares the plurality of output data PM generated in step S105 with the target data TM associated with the learning data LM from which each of the plurality of output data PM is generated, and calculates a plurality of evaluation values EV, and thus evaluates the result of the processing. The calculation unit 47 uses the average value of the plurality of evaluation values EV, as the final evaluation value EV indicating the degree of similarity SD. In step S109, the evaluation determination unit 46 calculates the coupling reference value RV, using the final evaluation value EV. In this embodiment, the evaluation determination unit 46 uses the degree of similarity SD for the coupling reference value RV. That is, when the final evaluation value EV is the mean squared error, the coupling reference value RV is the reciprocal of the evaluation value EV. When the PSNR, the MSE, or the SSIM is used as the final evaluation value EV, the value of the evaluation value EV is used as the coupling reference value RV.

Next, in step S110, the coupling condition table generating unit 55 executes the update of the coupling condition table 88. For example, when the coupling reference value RV calculated from the final evaluation value EV of the data processing sequence Sq1 shown in FIG. 7 is “0.800”, all the element sets that form the data processing sequence Sq1, each element set being made up of a pair of an input-side node element and an output-side node element, are extracted and the coupling reference value RV “0.800” is added to the field representing the extracted element set. In a field representing one element set in the coupling condition table 88, the average value ARV is prescribed as the final coupling reference value RV. That is, an average value ARVₙ of an element set calculated by adding a n-th coupling reference value RVn is calculated using the following equation (A):

$${ARV}_{n} = \{ {RV}_{n} + {ARV}_{n - 1} \times n \} / ( n + 1 ) . \qquad ( A )$$

RVn is the coupling reference value added at the n-th time. ARVₙ₋₁ is the average value prescribed in the field of the element set up to the previous time.

When the data processing sequence Sq1 includes a plurality of the same element sets, the coupling reference value RV may be added by the number of the same element sets or the coupling reference value RV may be added only once. In this embodiment, even when the one data processing sequence Sq1 includes a plurality of the same element sets, the coupling reference value RV is added only once. When the element set of the input-side node element and the output-side node element is expressed as [input-side node element, output-side node element], the data processing sequence Sq1 shown in FIG. 7 has element sets given below. The coupling condition table generating unit 55 adds the coupling reference value “0.800” to the field of each element set in the corresponding coupling condition table 88 and divides the result by (n+1), and thus calculates the average value ARV, as shown in FIG. 13, for each of the element sets given below. In this example, n=1 and therefore the average value ARV is “0.900”.

[input side=input layer in,output side=filter layer FtI]

[input side=filter layer FtI,output side=filter layer FtG]

[input side=filter layer FtG,output side=filter layer FtL]

[input side=input layer ConB,output side=filter layer FtL]

[input side=filter layer FtL,output side=output layer out]

As shown in FIG. 11, in step S111, the coupling condition table generating unit 55 determines whether the processing of steps S104 to S110 is executed for all the individuals IV of the one generation G or not. When the result of the determination in step S111 is “No”, the processor 25 selects one of the individuals IV for which the processing of steps S104 to S111 is not executed, of the individuals IV of the one generation G, and executes the processing of steps S104 to S111 again.

Meanwhile, when the result of the determination in step S111 is “Yes”, the coupling condition table generating unit 55 in step S113 determines whether an end condition is satisfied or not. The end condition is a condition that the processing routine of steps S103 to S111 is executed a predetermined prior number of times. The predetermined prior number of times is set, for example, to be such a number of times that the coupling reference value RV is added at least once, preferably twice or more, to all the fields of element sets in the coupling condition table 88. For example, the prior number of times is set in such a way that the processing routine of steps S104 to S111 is executed for individuals IV that are twice the number of element sets prescribed in the coupling condition table 88 or more.

As described above, the coupling condition table 88 is generated by using the degree of similarity SD generated using each of a plurality of data processing sequences Sq generated by the processing of step S104, for the coupling reference value RV, in such a way that node elements occur with a random probability. Thus, as the individual IV is generated using the coupling condition table 88, the probability that an element set with high suitability of coupling is included in the data processing sequence Sq can be increased.

When the end condition is not satisfied, the population generating unit 32 executes step S103 again in the next processing routine. Step S103 that is executed again is executed by replacing an individual IVₘ whose degree of similarity SD is at up to the J-th place from the bottom, of individuals IVₘ1 to IVₘ5 of a parent generation Gm generated in step S103 in the previous processing routine, with a new individual IVₙ. “J” is an integer equal to or greater than 1. That is, the population generating unit 32 copies the individual IV whose degree of similarity SD is not at up to the J-th place from the bottom. The population generating unit 32 performs at least one of crossover and mutation from an individual IV whose degree of similarity SD is not at up to the J-th place from the bottom, and thus generates a new individual IV. Thus, a new data processing sequence Sq having a different combination of sequence elements from the data processing sequence Sq generated up to the previous time can be easily generated. The processing from step S104 onward is executed again on the individual IVₙ generated by step S103 that is executed again.

Meanwhile, when the end condition is satisfied, the coupling condition table generating unit 55 in step S115 outputs the coupling condition table 88 as of when the end condition is satisfied, and thus causes the storage device 70 to store this coupling condition table 88.

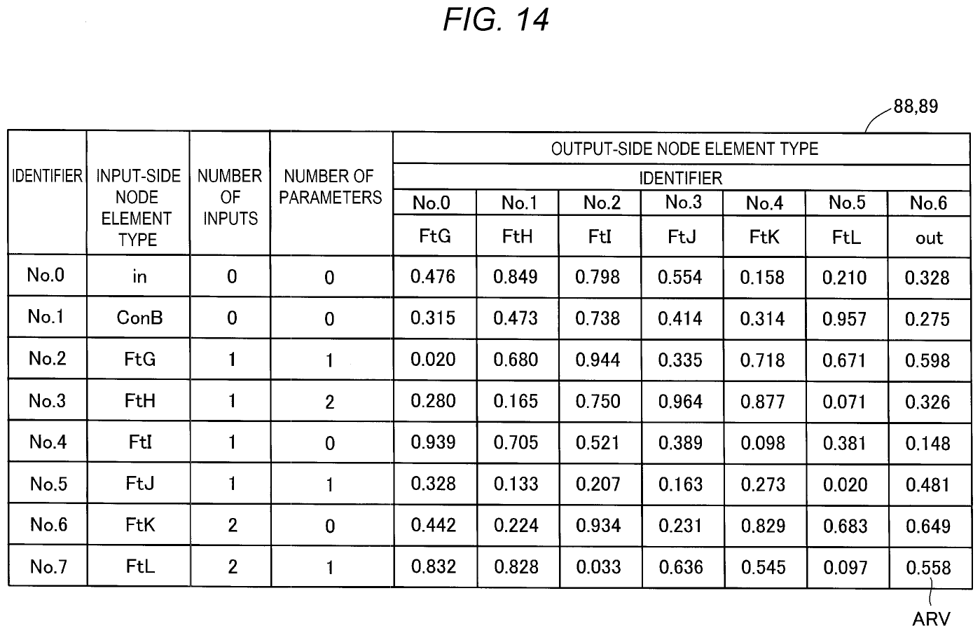

FIG. 14 shows the coupling condition table 88. When the generation processing for the coupling condition table 88 satisfies the end condition, the coupling condition table 88 as shown in FIG. 14 is stored in the storage device 70. For each element set prescribed in the coupling condition table 88, the average value ARV is prescribed as the final coupling reference value RV. In the specified generation processing, the sequence generating unit 30 refers to the coupling condition table 88, and the second-type gene table 82B shown in FIG. 4, as described later, in order to generate the data processing sequence Sq to be a candidate for the specified data processing sequence DSq. The coupling condition table 88 and the second-type gene table 82B, together with the type table 92 shown in FIG. 9, form the reference table 89.

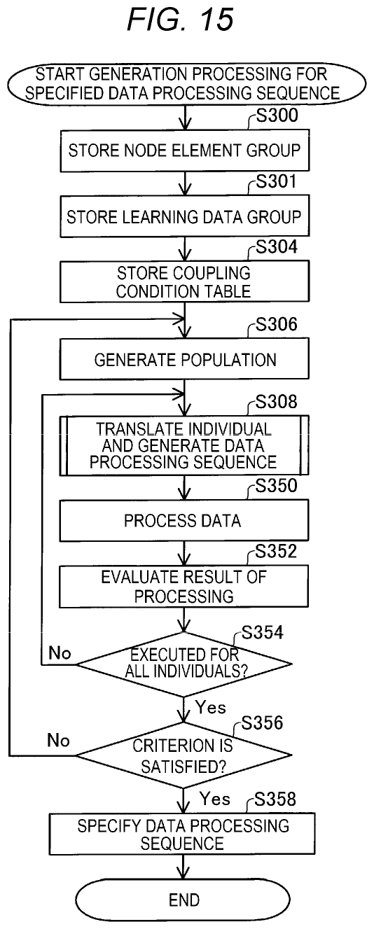

FIG. 15 is a detailed flowchart of the generation processing for the specified data processing sequence DSq in step S30 shown in FIG. 10. First, in step S300, the storage device 70 stores a plurality of filter layers and the input layer ConB as the node element group 80. Step S300 is executed by the user inputting, to the generating device 20, a node element such as a filter layer that is a candidate to be used in the specified data processing sequence DSq. When the generation processing for the specified data processing sequence DSq is executed by the same device as the generating device 20 executing the generation processing for the coupling condition table 88, the node element group 80 is already stored in the storage device 70 in step S100 in FIG. 11. Therefore, in this case, step S300 can be omitted.

In step S301, the storage device 70 stores the learning data group 74 formed of a plurality of learning sets SM. Step S301 is executed by the user inputting the learning data group 74 to the generating device 20. When the generation processing for the specified data processing sequence DSq is executed by the same device as the generating device 20 executing the generation processing for the coupling condition table 88 and the same learning data group 74 is used, the learning data group 74 is already stored in the storage device 70 and therefore step S301 can be omitted. Meanwhile, for example, when the learning data group 74 is generated based on a circuit board that is the same type as the circuit board which is the subject product from which the learning data group 74 used in the generation processing for the coupling condition table 88 is generated but that is produced, for example, in a different plant, or when the product from which the learning data group 74 is generated is different though the purpose of inspecting whether there is the defective part DA or not is the same, step S301 is executed.

In step S304, the storage device 70 stores the coupling condition table 88 generated in the generation processing for the coupling condition table 88. When the generation processing for the specified data processing sequence DSq is executed by the same device as the generating device 20 executing the generation processing for the coupling condition table 88, the coupling condition table 88 is already stored in the storage device 70. Therefore, in this case, step S304 can be omitted. The order of steps S300 to S304 is not limited to the foregoing order.

Next, in step S306, the population generating unit 32 generates a plurality of individuals IV and thus generates the population 84 of one generation. As described above, the individual IV is made up of a plurality of genes sequenced, each gene representing one of the type of the input layer and the filter layer, and the parameter set for the filter layer, as shown in FIG. 5. Step S306, where the data processing sequence Sq having the input layer, the filter layer, and the output layer as nodes is formed, is processing similar to step S103 in FIG. 11. That is, in step S306, the population 84 of one generation G is formed of a plurality of individuals IV1 to IV5. The generated population 84 is stored in the storage device 70.

Next, in step S308, the gene translation unit 34 selects one of the individuals IV1 to IV5, translates the selected one individual IV with reference to the second-type gene table 82B and the reference table 89 including the coupling condition table 88, and thus generates the data processing sequence Sq. That is, in step S308, the gene translation unit 34 allocates one of the input layer, which is one node, the filter layer, which is another node, and the parameter, to each gene forming the individual IV, with reference to the reference table 89 establishing the correspondence between the genes of the individual IV, and the nodes and the parameter, and thus generates the data processing sequence Sq corresponding to the individual IV. The generation of the data processing sequence Sq in step S308 is executed by the foregoing specified generation processing. Details of step S308 will be described later.

Next, in step S350, the data processing unit 40 performs data processing of a plurality of learning data LM, using the data processing sequence Sq generated in step S308, and thus generates a plurality of output data PM.

Next, in step S352, the evaluation determination unit 46 compares the plurality of output data PM generated in step S350 with the target data TM associated with the learning data LM from which each of the plurality of output data PM is generated, and calculates a plurality of evaluation values EV, and thus evaluates the result of the processing. The evaluation determination unit 46 uses the average value of the plurality of evaluation values EV that are calculated, as the final evaluation value EV indicating the degree of similarity SD between the output data PM and the target data TM.

Next, in step S354, the evaluation determination unit 46 determines whether the processing of steps S308 to S352 is executed for all the individuals IV of the one generation G or not. When the result of the determination in step S354 is “No”, the processor 25 selects one of the individuals IV for which the processing of steps S308 to S352 is not executed, of the individuals IV of the one generation G, and executes the processing of steps S308 to S352 again. That is, the data processing unit 40 performs data processing of the learning data LM, based on each of the plurality of data processing sequences Sq corresponding to the plurality of individuals IV forming the one generation G, and thus generates the output data PM for each of the plurality of data processing sequences Sq. The evaluation determination unit 46 then compares the plurality of output data PM generated for each of the plurality of data processing sequences Sq with the corresponding target data TM and thus calculates the evaluation value EV.

Meanwhile, when the result of the determination in step S354 is “Yes”, the specifying unit 49 of the evaluation determination unit 46 in step S356 determines whether there is a data processing sequence Sq that satisfies a predetermined criterion or not, using the plurality of evaluation values EV calculated for the current generation. Specifically, the specifying unit 49 of the evaluation determination unit 46 determines that there is a data processing sequence Sq that satisfies the criterion, when a highest degree of similarity SDmax, of the degrees of similarity SD indicated respectively by the plurality of evaluation values EV calculated based on the plurality of individuals IV of the current generation, is equal to or higher than a predetermined end threshold. That is, the process of step S356 is a process of specifying a data processing sequence Sq that satisfies the predetermined criterion, using the plurality of evaluation values EV corresponding respectively to the plurality of data processing sequences Sq.

When there is a data processing sequence Sq that satisfies the criterion, the specifying unit 49 of the evaluation determination unit 46 in step S358 outputs the data processing sequence Sq that satisfies the criterion, as the specified data processing sequence DSq. The outputted specified data processing sequence DSq is stored in the storage device 70.

Meanwhile, when there is no data processing sequence Sq that satisfies the criterion, the processing of steps S306 to S356 is executed as the next routine. That is, in step S306 in the next routine, the population 84 of the next generation is generated by a next generation generating process. In the next generation generating process, the population generating unit 32 replaces a subject data processing sequence Sq that is a data processing sequences Sq where one or more of the plurality of individuals IV generated in the previous routine have a low degree of similarity SD indicated by the evaluation value EV, with a new data processing sequence Sq having different sequence elements, and thus generates a sequence set. In this embodiment, the population generating unit 32 performs at least one of crossover and mutation from an individual IV whose degree of similarity SD is not at up to the J-th place from the bottom and is therefore high, of the plurality of individuals IV of the parent generation used in the previous routine, and thus replaces an individual IV whose degree of similarity SD is low, that is, at up to the J-th place from the bottom, with a new individual IV. In the next generation generating process, the population generating unit 32 copies the individual IV whose degree of similarity SD is not at up to the J-th place from the bottom and is therefore high. A new sequence set is formed of the copied individual IV and the individual IV newly generated using crossover or mutation. Then, the processing of steps S308 to S356 is executed again on the new sequence set. Since one or more individuals IV with a low degree of similarity SD are replaced with a new individual IV based on an individual IV with a high degree of similarity SD, as described above, the data processing sequence Sq with a low degree of similarity SD can be eliminated from the processing subjects. Thus, the data processing sequence Sq that satisfies the criterion can be specified more efficiently.

The next generation generating process includes a reference individual generating process of performing at least one of crossover and mutation with reference to the type table 92 shown in FIG. 9 and thus generating an individual IV of the next generation, and a normal individual generating process of performing at least one of crossover and mutation without referring to the type table 92 and thus generating an individual IV of the next generation. Which of the reference individual generating process and the normal individual generating process is to be executed for each individual IV in the next generation generating process is prescribed by the generation condition in the various condition table 93 shown in FIG. 2. This generation condition may be prescribed by an input from the user. Details of the reference individual generating process will be described later.

In the processing of steps S308 to S356 executed on the new sequence set, the evaluation value EV calculated in the previous routine or before is used for the same data processing sequence Sq as the data processing sequence Sq forming the sequence set used in the previous routine or before. Thus, the evaluation value EV need not be calculated again and therefore the processing efficiency of the specified data processing sequence generation processing can be improved.

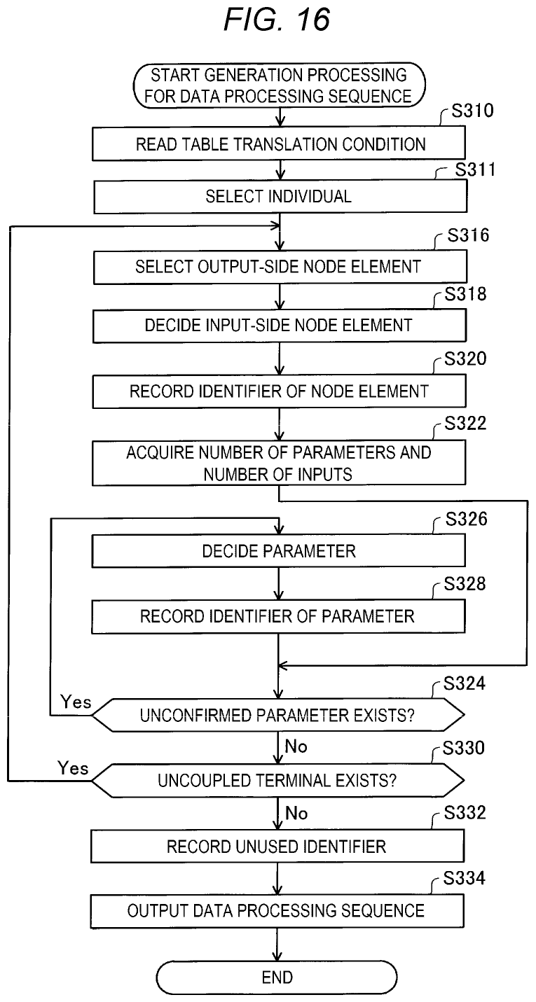

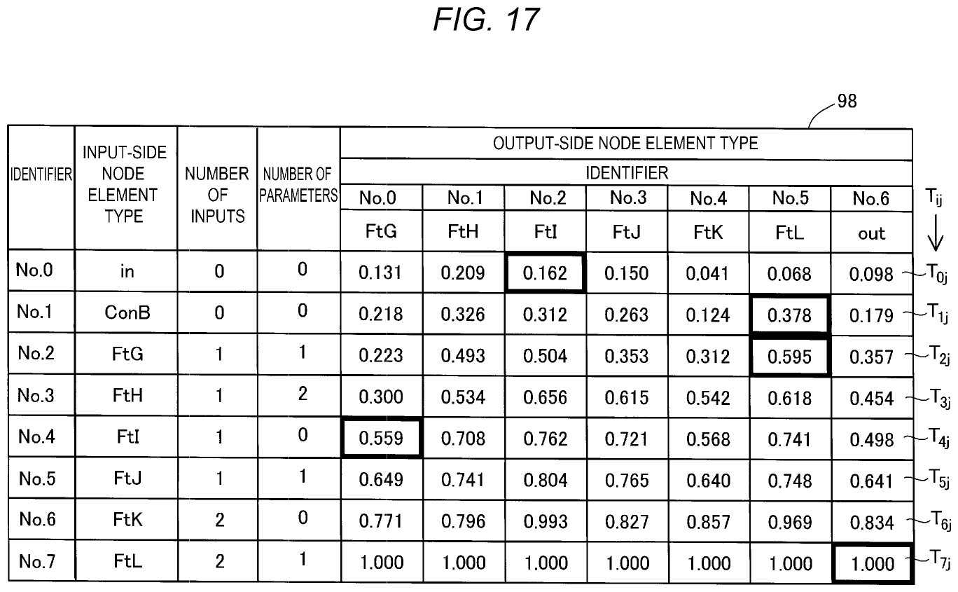

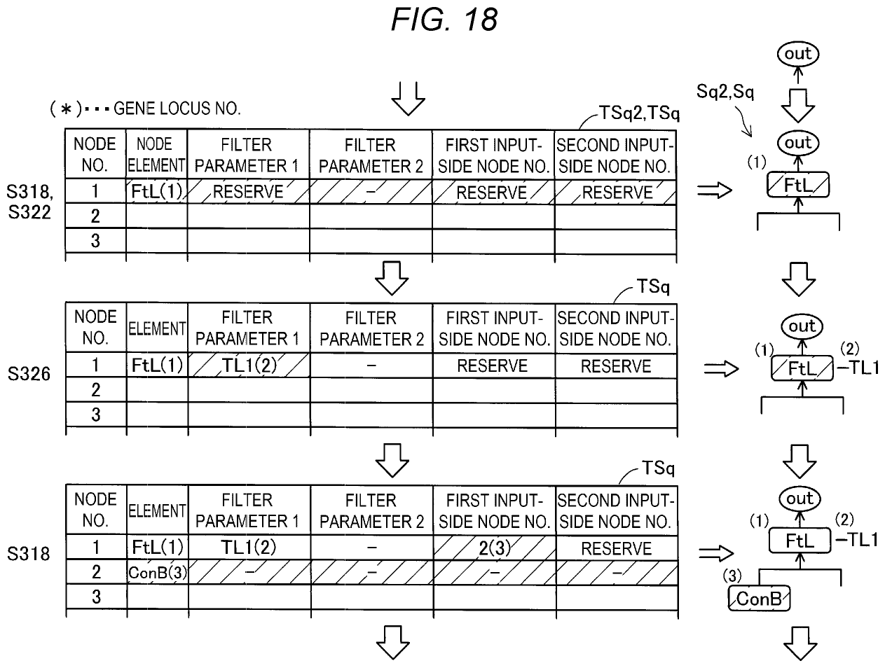

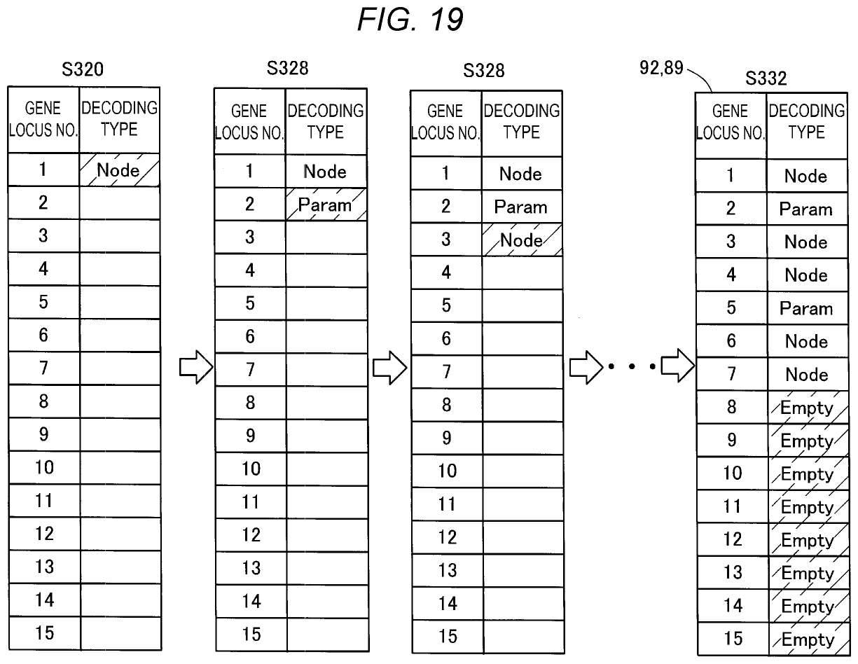

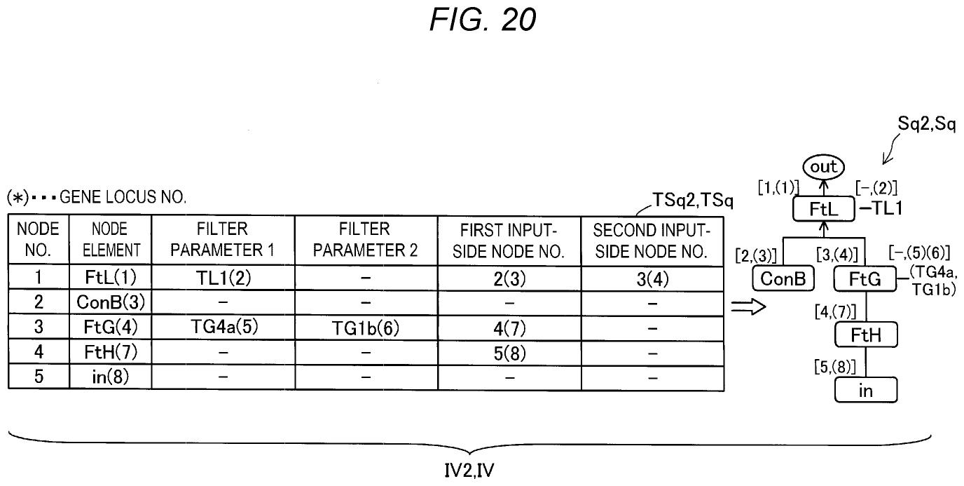

FIG. 16 is a detailed flowchart of step S308. FIG. 17 shows a threshold table 98 referred to in the processing of step S308. FIG. 18 is a first view illustrating step S308. FIG. 19 is a second view illustrating step S308. FIG. 20 shows a table-formatted data processing sequence TSq2 corresponding to the individual IV2. An element set in a bold-line part in FIG. 17 is an element set that occurs as the data processing sequence Sq of the individual IV2.

As shown in FIG. 16, first, the gene translation unit 34 in step S310 reads a table translation condition stored in the various condition table 93 in the storage device 70. The table translation condition is inputted by the user and defines a table referred to by the gene translation unit 34 in the processing of step S308. The table translation condition defines whether to refer to the first-type gene table 82A shown in FIG. 4, where the nodes of the filter layers FtG to FtL and the input layers in, ConB occur at an equal probability of occurrence, or the coupling condition table 88 shown in FIG. 8, where the probability of occurrence is set based on the suitability of coupling. When the table translation condition is not stored in the storage device 70, the gene translation unit 34 refers to the coupling condition table 88. The table showing the probability of occurrence of the parameter is the second-type gene table 82B shown in FIG. 4, no matter which table translation condition is set. In this embodiment, the table translation condition prescribes that the table referred to by the gene translation unit 34 is the coupling condition table 88.

When executing processing related to the generation of a sequence element of the data processing sequence Sq such as selecting an input-side node element or acquiring the number of parameters in the processing from step S316 onward, the sequence generating unit 30 updates the table-formatted data processing sequence TSq shown in FIG. 6 along with the execution of the processing.

In step S311, the gene translation unit 34 selects an unprocessed one of the individuals IV1 to IV5 forming the current generation G stored in the storage device 70. A case where the individual IV2 shown in FIG. 5 is selected is described below.

Next, in step S316, the gene translation unit 34 selects one uncoupled output-side node element to which an input-side node element is not coupled according to a coupling rule. In this embodiment, the data processing sequence Sq is generated in order from the output side to the input side, as shown in FIG. 6. Therefore, in the first routine, the output layer out is set as the end-point node. Thus, in step S316 in the first routine, the gene translation unit 34 selects the output layer out as the output-side node element. The coupling of node elements is executed according to a predetermined coupling rule as described above. For example, the gene translation unit 34 sets the output layer out as the end-point node, which is the output-side end, and couples a node to the input side in the order of the sequence of the genes of the individual IV, and thus generates the data processing sequence Sq.

Following step S316, the gene translation unit 34 in step S318 determines an input-side node to be coupled to the output-side node, which is a coupling point, based on the coupling condition table 88 and the genes of the selected individual IV. Specifically, the gene translation unit 34 generates the data processing sequence Sq in such a way as to achieve a relationship of probability such that the probability of coupling to the output-side node is higher for an input-side node having a higher average value ARV as the coupling reference value RV in relation to the output-side node, using the coupling condition table 88. The data processing sequence Sq having the above relationship of probability is generated by the selection of an input-side node element in relation to an output-side node element according to a node selection algorithm given below.

### Selection Algorithm for Input-Side Node Element

The preconditions of the node selection algorithm are as follows.

VG is the numeric value of a gene. VG is a numeric value up to the third place after the decimal point, of values from 0 to 1.

j is the identifier of an output-side node element. j is expressed by an integer equal to or greater than 0. A different value is allocated from one output-side node element to another. In this embodiment, as the identifier j, 0 to 6 are allocated in the order of the output-side node elements FtG to out.

i is the identifier of an input-side node element to be coupled. i is expressed by an integer equal to or greater than 0 and expressed as an identifier of 0 to N_{f}−1. N_{f} is the number of candidates for the input-side node element. In this embodiment, the number of input-side node elements N_{f} is 8, as shown in FIG. 8. As the identifier i, 0 to 7 are allocated in the order of the input-side node elements in to FtL.

tᵢⱼ is the value of an element set in the coupling condition table 88. tᵢⱼ is the average value ARV shown in FIG. 14. The subscript “ij” is an identifier to specify an element set and corresponds to the identifiers of the input-side node element and the output-side node element. For example, t₁₂ represents 0.738, which is the average value ARV of the element set of the input layer ConB as the input-side node element and the filter layer FtI as the output-side node element in FIG. 14.

In the node selection algorithm, a probability Pᵢⱼ that the input-side node element i is coupled to the input side of the output-side node element j is calculated by an equation (1) given below.

$$[ Math . 1 ]$$

$${P}_{ij} = {t}_{ij} / ( {\sum }_{k = 0}^{{N}_{f} - 1} {t}_{kj} ) \qquad ( 1 )$$

In the node selection algorithm, an upper threshold Tᵢⱼ of the numeric range of the gene where the input-side node element i is coupled to the output-side node element j is calculated, using an equation (2) given below. A lower threshold is set to be the upper threshold of the input-side node element corresponding to the immediately previous identifier. When there is no immediately previous identifier, the lower threshold is set to be 0.

$$[ Math . 2 ]$$

$${T}_{ij} = {\sum }_{k = 0}^{i} {P}_{kj} = ( {\sum }_{k = 0}^{i} {t}_{kj} ) / ( {\sum }_{k = 0}^{{N}_{f} - 1} {t}_{kj} ) . \qquad ( 2 )$$

As the numeric range representing the range of occurrence of the gene is determined using the above node selection algorithm, the data processing sequence Sq is generated in such a way that the ratio of the average values ARV, which are the coupling reference values of the input-side node elements that are candidates to be coupled to the same output-side node element is the ratio of the probabilities of coupling to the output-side node element.

In this embodiment, the gene translation unit 34 selects an input-side node element in the following manner according to the value VG of the gene, using the node selection algorithm. An inequality sign may be changed to include or exclude an equality sign as long as the value VG of the gene is included in one of the ranges of occurrence. That is, the gene translation unit 34 selects:

the input layer in, which is the input-side node element No.0, when 0≤VG<T₀ⱼ;

the input layer ConB, which is the input-side node element No. 1, when T₀ⱼ≤VG≤Tᵢⱼ;

the filter layer FtG, which is the input-side node element No. 2, when Tᵢⱼ<VG≤T₂ⱼ;

the filter layer FtH, which is the input-side node element No. 3, when T₂ⱼ<VG≤T₃ⱼ;

the filter layer FtI, which is the input-side node element No. 4, when T₃ⱼ<VG≤T₄ⱼ;

the filter layer FtJ, which is the input-side node element No. 5, when T₄ⱼ<VG≤T₅ⱼ;

the filter layer FtK, which is the input-side node element No. 6, when T₅ⱼ<VG≤T₆ⱼ; and

the filter layer FtL, which is the input-side node element No. 7, when T₆ⱼ<VG≤T₇ⱼ.

The threshold table 98 shown in FIG. 17 is a table where the upper threshold Tᵢⱼ calculated by the gene translation unit 34 using the node selection algorithm in relation to the coupling condition table 88 shown in FIG. 14 is prescribed for the element sets. The gene translation unit 34 may generate the threshold table 98 shown in FIG. 17 before the processing of step S308 is started, or may generate at least a necessary part to select an input-side node element, of the threshold table 98 shown in FIG. 17, when executing step S318.

In step S318 shown in FIG. 16, the gene translation unit 34 compares the upper threshold Tᵢⱼ described in the column of the output layer out as the output-side node element in the threshold table 98, with “0.921”, which is a value VG1 of the gene of gene locus No.1 of the individual IV2. Thus, the gene translation unit 34 selects the filter layer FtL corresponding to the value VG1, as the input-side node element. In step S318, the gene translation unit 34 also sets the filter layer FtL as the node element corresponding to node No.1, as shown in the illustration at the top of FIG. 18. In the table-formatted data processing sequence TSq2, the number in parentheses given to the filter layer FtL indicates the gene locus number of the individual IV2 in order to facilitate understanding.

As shown in FIG. 16, in step S320 following step S318, the type table generating unit 36 associates an identifier identifying that it is a node element, with a gene locus where a corresponding gene is arranged, and thus records the identifier in the type table 92. In this embodiment, the type table generating unit 36 records an identifier “Node” indicating a node element as a decoding type in association with gene locus No.1 where the corresponding gene that is the source of translation is arranged, in the type table 92, as shown at the leftmost part of FIG. 19.

In step S322 following step S320, the gene translation unit 34 acquires the number of parameters and the number of inputs of the input-side node element selected in step S318, referring to the coupling condition table 88. In step S322, the gene translation unit 34 sets a reservation code in the table-formatted data processing sequence TSq2 shown in FIG. 18 according to the number of parameters and the number of inputs that are acquired. Specifically, the number of parameters of the filter layer FtL set for node No.1 is “1” and the number of inputs thereof is “2”. Therefore, the gene translation unit 34 sets a reservation code for the filter parameter 1, the first input-side node number, and the second input-side node number corresponding to node No.1 in the table-formatted data processing sequence TSq.

As shown in FIG. 16, in step S324, the gene translation unit 34 determines whether an unconfirmed parameter exists or not. In step S324, the gene translation unit 34 compares the number of parameters acquired in step S322 with the number of parameters translated to sequence elements, and determines that an unconfirmed parameter exists when these numbers of parameters do not coincide with each other. Specifically, the gene translation unit 34 determines that an unconfirmed parameter exists when a reservation code is set for at least one of the items of the filter parameter 1 and the filter parameter 2 in the table-formatted data processing sequence TSq2. In the example shown in FIG. 18, a reservation code is set for the filter parameter 1 corresponding to the filter layer FtL. Therefore, the gene translation unit 34 determines that an unconfirmed parameter exists, and executes step S326.

In step S326 shown in FIG. 16, the gene translation unit 34 determines a parameter, using the second-type gene table 82B prescribing the range of occurrence of the parameter, and the value of the gene of the source of translation. For example, as shown in the middle of FIG. 18, the parameter value TL1 corresponding to the value VG2 of the gene of gene locus No.2 is determined as the parameter of the filter layer FtL of node No.1.

Next, in step S328, the type table generating unit 36 associates an identifier identifying that it is a parameter, with a gene locus number where a corresponding gene is arranged, and thus records the identifier in the type table 92. In this embodiment, the type table generating unit 36 records an identifier “Param” indicating a parameter as a decoding type in association with gene locus No.2 where the corresponding gene that is the source of translation is arranged, in the type table 92, as shown in the second illustration from the left in FIG. 19.

As shown in FIG. 16, following step S328, the gene translation unit 34 executes step S324 again. As shown in the middle of FIG. 18, a reservation code is not set for the filter parameters 1, 2 of the filter layer FtL of node No.1. In this case, the gene translation unit 34 determines that there is no unconfirmed parameter, and executes step S330 shown in FIG. 16. In step S330, the gene translation unit 34 determines whether an uncoupled node exists in the data processing sequence Sq in the generating process or not. The gene translation unit 34 determines that there is no uncoupled node when all the input-side nodes are the input layers in, ConB.

When it is determined that an uncoupled node exists, the gene translation unit 34 executes step S316 again. In the example shown in FIG. 18, there are two uncoupled input-side nodes which take the filter layer FtL of node No.1 as the output-side node element and for which a reservation code is prescribed. Therefore, based on the coupling rule, the gene translation unit 34 selects input-side node No.1 associated with node No. 1 as the uncoupled node. In the next step S318, the gene translation unit 34 determines the node element of node No. 2 as the input layer ConB, referring to a value VG3 of the gene of gene locus No. 3 and the threshold table 98.

Meanwhile, when the gene translation unit 34 has determined that there is no uncoupled node, the type table generating unit 36 in step S332 associates an unused identifier that is not used for the translation with a gene locus where a corresponding gene is arranged, and thus records the identifier in the type table 92. For example, in the generation of the data processing sequence Sq2 using the individual IV2, the genes of gene locus Nos. 8 to 15 are not used for the translation and therefore an identifier “Empty” is recorded for gene locus Nos. 8 to 15, as shown in the type table 92 on the right side in FIG. 19.

As shown in FIG. 16, in step S334 following step S332, the gene translation unit 34 outputs the translated data processing sequence Sq. For example, the data processing sequence Sq to be outputted may be the table-formatted data processing sequence TSq as shown in FIG. 20. The outputted data processing sequence Sq is stored in the storage device 70. Thus, the generating process of step S308 ends.

When there is an uncoupled node or an unconfirmed parameter even if all the genes of the individual IV are used, the sequence generating unit 30 executes end processing, describe below. In the end processing, when there is an uncoupled node, the gene translation unit 34 sets all the input layers in for the uncoupled node. In the end processing, when there is an unconfirmed parameter, the gene translation unit 34 sets a predetermined specified parameter. In the end processing, when an uncoupled node or an unconfirmed parameter is set, there is no gene of the source of translation and therefore the recording of a type code in the type table 92 is not executed.

Details of step S306 executed from the second routine onward in the flowchart shown in FIG. 15 will now be described. In step S306 from the second routine onward, the population 84 is newly generated, using at least one of the reference individual generating process and the normal individual generating process according to a predetermined generation condition. In the normal individual generating process, which is a generating process based on a normal genetic algorithm, at least one of crossover and mutation is performed and the individual IV of the population 84 is generated without referring to the type table 92. In the reference individual generating process, at least one of a crossover point and a gene to mutate is determined and at least one of crossover and mutation is performed with reference to the type table 92. In the reference individual generating process, the process of executing mutation is also referred to as a type-referenced mutation process, and the process of executing crossover is also referred to as a type-referenced crossover process. In this way, when generating the individual IV of the next generation, at least one of crossover and mutation is performed in relation to the individual IV in the previous routine, with reference to the type table 92. Thus, the individual IV of the next generation can be easily generated, paying attention to the parameter of the filter layer. For example, causing the mutation of a gene representing the parameter of the individual IV of the previous routine enables easy generation of the individual IV of the next generation where only the parameter is changed.

In the reference individual generating process, when generating the individual IV by mutation, the population generating unit 32 performs mutation of a specified gene associated with the parameter as the decoding type, of a plurality of genes of the individual IV that is a subject of mutation. In the type-referenced crossover process, when generating the individual IV by crossover, for example, by single-point crossover, the population generating unit 32 sets a crossover point between the specified gene and the next gene, of a plurality of genes of one of the individuals IV from which the new individual IV is generated, and crossover is thus performed. The type-referenced mutation process will now be described, using an illustration.

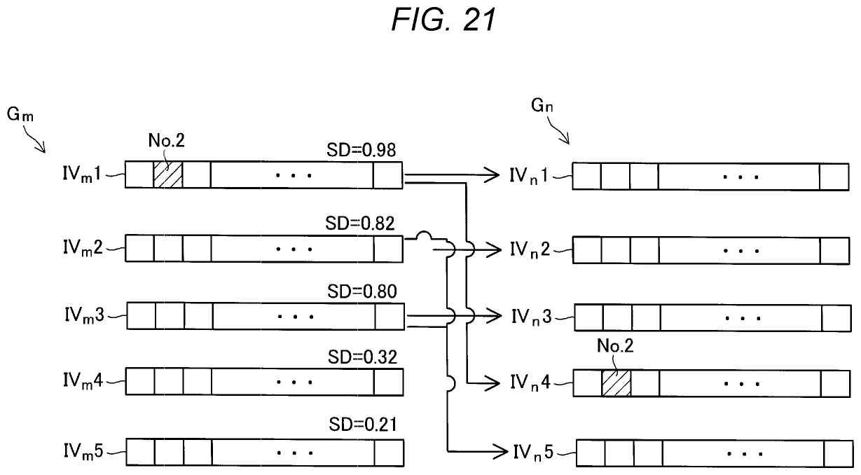

FIG. 21 explains the type-referenced mutation process in the reference individual generating process. In this embodiment, the generation condition includes a condition that, for an individual IVₘ whose degree of similarity SD is at up to the second place from the bottom, a new individual IVₙ is generated by mutation or crossover from another individual IVₘ with a high degree of similarity SD. In this embodiment, the generation condition also includes a condition that, when performing mutation, the type-referenced mutation process in the reference individual generating process is executed for an individual IVₘ from which the data processing sequence Sq with the degree of similarity SD at up to the N-th place from the top is generated, and the normal individual generating process is executed for the other individuals IVₘ. Thus, for the data processing sequence Sq whose degree of similarity SD is at up to the N-th place from the top, whether the criterion is satisfied or not can be evaluated simply by changing the parameter of the filter layer. “N” is an integer equal to or greater than 1. The upper limit of “N” is the number of individuals IV to be copied. In this embodiment, “N” is set to be “1”. That is, in this embodiment, the type-referenced mutation process is executed only for an individual IVₘ1 with the best degree of similarity SD, of the individual IVₘ of the parent generation Gₘ. Thus, for the best individual IVₘ1, whether the criterion is satisfied or not can be evaluated simply by changing the parameter value. The generation condition also includes a condition that the normal individual generating process is executed when performing crossover. These generation conditions are set, for example, by the user. Based on the foregoing generation conditions, for individuals IVₘ4, IVₘ5 at up to the second place from the bottom, of individuals IVₘ1 to IVₘ5 of the parent generation Gₘ, the population generating unit 32 generates new individuals IVₙ4, IVₙ5, based on the other individuals IVₘ1 to IVₘ3 at up to the third place from the top, and thus replaces the individuals IVₘ4, IVₘ5. The population generating unit 32 also copies the individuals IVₘ1 to IVₘ3 whose degree of similarity SD is at up to the third place from the top, and uses the copied individuals as individuals IVₙ1 to IVₙ3 of the current generation Gn.

The population generating unit 32 also executes the type-referenced mutation process for the individual IVₘ1 of the parent generation with the highest degree of similarity SD and thus generates an individual IVₙ4 of the current generation Gn. Specifically, the population generating unit 32 specifies a gene locus associated with the identifier “Param” as the decoding type with reference to the type table 92. In this embodiment, the identifier “Param” is associated with gene locus No.2. In this case, the population generating unit 32 performs the mutation of the gene of gene locus No.2 of the individual IVₘ1 of the parent generation Gₘ and thus generates the individual IVₙ4 of the current generation Gn.

The population generating unit 32 sets an arbitrary position as a crossover point for the individual IVₘ2 and the individual IVₘ3 of the parent generation Gₘ without referring to the type table 92 and executes crossover, and thus generates the individual IVₙ5 of the current generation Gn.

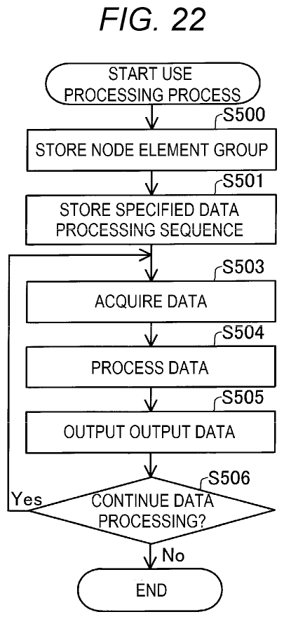

FIG. 22 is a flowchart of the use processing process of step S50 shown in FIG. 10. First, in step S500, the storage device 70 stores the node element group 80 accepted by the input unit 150. The node element group 80 stored in the use processing process may be only the node element used in the specified data processing sequence DSq. When the use processing process and step S30 in FIG. 10 are executed by the same generating device 20, step S500 can be omitted and the node element group 80 stored in the storage device 70 in step S100 can be used.

Next, in step S501, the storage device 70 stores the specified data processing sequence DSq specified in step S358 in FIG. 15. The order of steps S500 and S501 is not limited to the foregoing order.

Next, in step S503, the data processing unit 40 reads out and thus acquires data that is stored in the storage device 70 via the input unit 150 and that is a subject of data processing of the specified data processing sequence DSq. The data acquired in step S503 is, for example, data of the same type as the learning data LM, and in this embodiment, a picked-up image of a subject. When there are a plurality of data to be a subject of data processing, the data processing unit 40 may read out one of the plurality of data stored in advance in the storage device 70 and thus may acquire the data, or may acquire data to be a subject each time from the sensor device 200 at the timing of executing data processing, described later.

Next, in step S504, the data processing unit 40 executes data processing using the specified data processing sequence DSq, on the data acquired in step S503. That is, data processing is executed on the image that is the data, according to the order and the parameter value represented by the specified data processing sequence DSq.

Next, in step S505, the output data PM, which is the data after the data processing using the specified data processing sequence DSq, is outputted. Step S505 is executed, for example, as the output data PM is stored in the storage device 70 or displayed on the display unit 160.

Next, in step S506, the data processing unit 40 determines whether to continue the data processing using the specified data processing sequence DSq or not. For example, when information indicating the continuation of the data processing is inputted by the user or when unprocessed data to be a subject of the data processing is left in the storage device 70, the data processing unit 40 determines that the data processing is to be continued. When continuing the data processing, the processing from step S503 onward is executed again. Meanwhile, when information indicating the end of the data processing is inputted by the user or when unprocessed data to be a target of the data processing is not left in the storage device 70, the data processing unit 40 ends the use processing process.

According to the foregoing embodiment, the genes forming the individual IV represent the parameter that is set for the filter layer in addition to the nodes of the input layer in and the filter layer, as shown in FIGS. 5 and 7. Thus, in the process of generating a population, the individual IV can be generated, paying attention to the parameter. For example, the genes can be used, corresponding to the number of parameters that varies depending on the type of the filter layer. Also, for example, the individual IV representing the data processing sequence Sq where only the parameter of a specified filter layer is changed can be easily generated. Also, according to the foregoing embodiment, since a decoding type is associated with each gene in the type table generating process executed in steps S320, S328, and S332 shown in FIG. 16, the generation of the individual IV paying attention to the parameter can be easily executed in step S306 shown in FIG. 15 by referring to the type table 92. Particularly, as shown in FIG. 21, the individual IVₙ4 is generated by the mutation of the gene associated with the parameter as the decoding type. Therefore, the individual IV where only the parameter of the filter layer is changed from the individual IVₘ1 that is the subject of mutation can be easily generated. Thus, the data processing sequence Sq where only the parameter value is changed can be generated and whether the criterion is satisfied or not can be evaluated.

According to the foregoing embodiment, in the next generation generating process, at least one of the reference individual generating process and the normal individual generating process is executed, based on the predetermined generation condition. Thus, setting the generation condition for the generation of the data processing sequence Sq to be a candidate for the specified data processing sequence DSq that satisfies the criterion enables the generation of the data processing sequence generated while paying attention to the parameter and the generation of the data processing sequence Sq generated without paying attention to the parameter. That is, setting the generation condition enables the optimization of the data processing sequence Sq by adjusting the parameter, and the optimization of the data processing sequence Sq by generating various data processing sequences.

### B. Other Embodiments

### B-1. Other Embodiment 1

In the foregoing embodiment, the range of occurrence in the second-type gene table 82B is set for each parameter in such a way that a plurality of parameter values occur evenly, as shown in FIG. 4. However, this is not limiting. The range of occurrence may be set unevenly. The second-type gene table 82B may also be, for example, a table where the value VG of a gene and the value VA of a parameter are defined by a function. For example, when the value VA of the parameter can be set within a range of 0.5 or greater and 1.5 or smaller and the value VG of the gene is set within a range of 0 or greater and 1 or smaller, the value VA may be expressed by an equation (B) given below, which is a linear function. The second-type gene table 82B may also be prescribed by a function to perform nonlinear transformation.

$$VA = VG + 0.5 \qquad ( B )$$

### B-2. Other Embodiment 2

In the foregoing embodiment, the gene translation unit 34 determines the parameter of the filter layer each time, referring to the second-type gene table 82B and the value VG of the gene that is the source of translation, as in step S326 shown in FIG. 16. However, this is not limiting. For example, the gene translation unit 34 may store the value of the gene from which the parameter is generated, that is, the source of translation, in the storage device 70 in advance, and may determine the value of the parameter at a time with reference to the second-type gene table 82B and the value VG of the gene after “No” is given as the result of the determination in step S330.

### C. Other Aspects

The present disclosure is not limited to the foregoing embodiment and can be implemented according to various aspects without departing from the spirit and scope of the present disclosure. For example, the present disclosure can be implemented according to the aspects given below. A technical feature in the foregoing embodiment corresponding to a technical feature in the aspects described below can be replaced or combined where appropriate in order to solve a part or all of the problems of the present disclosure or in order to achieve a part or all of the effects of the present disclosure. Such a technical feature can be deleted where appropriate unless described as essential in the specification.
(1) According to a first aspect of the present disclosure, a method for generating a data processing sequence is provided. The data processing sequence has an input layer, a filter layer, and an output layer, as nodes. The method for generating the data processing sequence includes: (a) generating a plurality of individuals in which a plurality of genes for expressing one of a type of the input layer and the filter layer, and a parameter set for the filter layer, forming a data processing sequence having an input layer, a filter layer, and an output layer, as nodes, are sequenced, and thus generating a population of one generation; (b) allocating one of the input layer, which is one of the nodes, the filter layer, which is another one of the nodes, and the parameter, to each of the genes forming the individual, with reference to a reference table establishing a correspondence between the gene, and the node and the parameter, and thus generating the data processing sequence corresponding to the individual; (c) performing data processing of subject data for each of a plurality of the data processing sequences expressed by the population, then comparing output data outputted for each of the data processing sequences, and target data to be a target associated with the subject data, and calculating an evaluation value indicating a degree of similarity between the output data and the target data; and (d) specifying the data processing sequence that satisfies a predetermined criterion, using a plurality of the evaluation values corresponding to the plurality of the data processing sequences, respectively. According to this aspect, the genes forming the individual represent the parameter set for the filter layer in addition to the nodes of the input layer and the filter layer. Thus, in the (a), the individual can be generated, paying attention to the parameter. For example, the genes can be used, corresponding to the number of parameters that varies depending on the type of the filter layer.
(2) In the method according to the above aspect, the predetermined criterion in the (d) may be a condition that the degree of similarity having a highest value of a plurality of the degrees of similarity indicated respectively by a plurality of the evaluation values is equal to or higher than a predetermined end threshold. The (a) may include a next generation generating process of performing at least one of crossover and mutation from the individual whose degree of similarity is high and thus replacing one or more of the individuals whose degree of similarity is low, of the plurality of individuals generated by a previous routine, with the individual that is new, and copying the individual whose degree of similarity is high and thus generating the population of a next generation used in a routine of this time, when the predetermined criterion is not satisfied in the (d). According to this aspect, since one or more individuals with a low degree of similarity are replaced with a new individual based on an individual with a high degree of similarity, a data processing sequence with a low degree of similarity can be eliminated from processing subjects. Thus, a data processing sequence that satisfies the criterion can be specified more efficiently.
(3) In the method according to the above aspect, the (b) may include a type table generating process of generating a type table where a decoding type for distinguishing the node represented by the gene and the parameter is associated with each of the genes forming the individual from which the data processing sequence is generated. The reference table may include the type table. According to this aspect, since a decoding type is associated to each gene by the type table generating process, the generation of an individual paying attention to the parameter can be easily executed in the (a) by referring to the type table.
(4) In the method according to the above aspect, the decoding type may include a type for distinguishing the gene that is unused and that is not used for the generation of the data processing sequence, in addition to distinguishing the node and the parameter. The type table generating process may generate the type table where the decoding type for distinguishing the node represented by the gene, the parameter, and the unused gene is associated with each of the genes forming the individual from which the data processing sequence is generated. According to this aspect, an unused gene that is not used for the generation of the data processing sequence can be easily specified, based on the type table.
(5) In the method according to the above aspect, the next generation generating process may include a reference individual generating process of determining at least one of a crossover point and the gene to perform the mutation and performing at least one of the crossover and the mutation with reference to the type table. According to this aspect, when generating an individual of the next generation, at least one of crossover and mutation from the individual in the previous routine is performed with reference to the type table. Thus, the generation of an individual of the next generation paying attention to the parameter can be easily performed.
(6) In the method according to the above aspect, the reference individual generating process may include a type-referenced mutation process of performing the mutation of the gene associated with the parameter as the decoding type, of the plurality of genes of the individual that is a subject of the mutation, when generating the individual by the mutation. According to this aspect, the mutation of the gene associated with the parameter as the decoding type is performed to generate an individual. Therefore, the individual where only the parameter of the filter layer is changed from the individual that is the subject of mutation can be easily generated. Thus, the data processing sequence where only the parameter value is changed can be generated and whether the criterion is satisfied or not can be evaluated.
(7) In the method according to the above aspect, the next generation generating process may further include a normal individual generating process of performing at least one of crossover and mutation without referring to the type table, and thus generating the individual. The next generation generating process may execute at least one of the reference individual generating process and the normal individual generating process, based on a predetermined generation condition. According to this aspect, setting the generation condition for the generation of a data processing sequence to be a candidate for the data processing sequence that satisfies the criterion enables the generation of a data processing sequence generated while paying attention to the parameter and the generation of a data processing sequence generated without paying attention to the parameter.
(8) In the method according to the above aspect, the generation condition may also be a condition that the type-referenced mutation process is executed for the individual from which the data processing sequence with the degree of similarity at up to an N-th place from the top, N being an integer equal to or greater than 1, is generated, of the individuals whose degree of similarity is high among the plurality of individuals generated by the previous routine. According to this aspect, for the data processing sequence whose degree of similarity is at up to the N-th place from the top, whether the criterion is satisfied or not can be evaluated simply by changing the parameter of the filter layer.
(9) According to a second aspect of the present disclosure, a device for generating a data processing sequence is provided. The data processing sequence has an input layer, a filter layer, and an output layer, as nodes. The device for generating the data processing sequence includes: a population generating unit that generates a plurality of individuals in which a plurality of genes for expressing one of a type of the input layer and the filter layer, and a parameter set for the filter layer, forming a data processing sequence having an input layer, a filter layer to perform data processing, and an output layer, as nodes, are sequenced, and thus generates a population of one generation; a gene translation unit that allocates one of the input layer, which is one of the nodes, the filter layer, which is another one of the nodes, and the parameter, to each of the genes forming the individual, with reference to a reference table establishing a correspondence between the gene, and the node and the parameter, and thus generates the data processing sequence corresponding to the individual; a calculation unit that performs data processing of subject data for each of a plurality of the data processing sequences expressed by the population, then compares output data outputted for each of the data processing sequences, and target data to be a target associated with the subject data, and calculates an evaluation value indicating a degree of similarity between the output data and the target data; and a specifying unit that specifies the data processing sequence that satisfies a predetermined criterion, using a plurality of the evaluation values corresponding to the plurality of the data processing sequences, respectively. According to this aspect, the genes forming the individual represent the parameter set for the filter layer in addition to the nodes of the input layer and the filter layer. Thus, the population generating unit can generate the individual, paying attention to the parameter. For example, the genes can be used, corresponding to the number of parameters that varies depending on the type of the filter layer.
(10) According to a third aspect of the present disclosure, a computer program for causing a computer to execute generation of a data processing sequence is provided. The data processing sequence has an input layer, a filter layer, and an output layer, as nodes. The computer program includes: (a) a function of generating a plurality of individuals in which a plurality of genes for expressing one of a type of the input layer and the filter layer, and a parameter set for the filter layer, forming a data processing sequence having an input layer, a filter layer to perform data processing, and an output layer, as nodes, are sequenced, and thus generating a population of one generation; (b) a function of allocating one of the input layer, which is one of the nodes, the filter layer, which is another one of the nodes, and the parameter, to each of the genes forming the individual, with reference to a reference table establishing a correspondence between the gene, and the node and the parameter, and thus generating the data processing sequence corresponding to the individual; (c) a function of performing data processing of subject data for each of a plurality of the data processing sequences expressed by the population, then comparing output data outputted for each of the data processing sequences, and target data to be a target associated with the subject data, and calculating an evaluation value indicating a degree of similarity between the output data and the target data; and (d) a function of specifying the data processing sequence that satisfies a predetermined criterion, using a plurality of the evaluation values corresponding to the plurality of the data processing sequences, respectively. According to this aspect, the genes forming the individual represent the parameter set for the filter layer in addition to the nodes of the input layer and the filter layer. Thus, the function (a) can generate the individual, paying attention to the parameter. For example, the genes can be used, corresponding to the number of parameters that varies depending on the type of the filter layer.

The present disclosure can be implemented according to various other aspects than the above aspects. For example, the present disclosure can be implemented in the form of a non-transitory storage medium storing a computer program.

What is claimed is:

1. A method for generating a data processing sequence,
the data processing sequence having an input layer, a filter layer, and an output layer, as nodes,
the method comprising:
(a) generating a plurality of individuals in which a plurality of genes for expressing one of a type of the input layer and the filter layer, and a parameter set for the filter layer, are sequenced, and thus generating a population of one generation;
(b) allocating one of the input layer, which is one of the nodes, the filter layer, which is another one of the nodes, and the parameter, to each of the genes forming the individual, with reference a to reference table establishing a correspondence between the gene, and the node and the parameter, and thus generating the data processing sequence corresponding to the individual;
(c) performing data processing of subject data for each of a plurality of the data processing sequences expressed by the population, then comparing output data outputted for each of the data processing sequences, and target data to be a target associated with the subject data, and calculating an evaluation value indicating a degree of similarity between the output data and the target data; and
(d) specifying the data processing sequence that satisfies a predetermined criterion, using a plurality of the evaluation values corresponding to the plurality of the data processing sequences, respectively.

2. The method according to claim 1, wherein
the predetermined criterion in the (d) is a condition that the degree of similarity having a highest value of a plurality of the degrees of similarity indicated respectively by a plurality of the evaluation values is equal to or higher than a predetermined end threshold, and
the (a) includes a next generation generating process of performing at least one of crossover and mutation from the individual whose degree of similarity is high and thus replacing one or more of the individuals whose degree of similarity is low, of the plurality of individuals generated by a previous routine, with the individual that is new, and copying the individual whose degree of similarity is high and thus generating the population of a next generation used in a routine of this time, when the predetermined criterion is not satisfied in the (d).

3. The method according to claim 2, wherein
the (b) includes a type table generating process of generating a type table where a decoding type for distinguishing the node represented by the gene and the parameter is associated with each of the genes forming the individual from which the data processing sequence is generated, and
the reference table includes the type table.

4. The method according to claim 3, wherein
the decoding type includes a type for distinguishing the gene that is unused and that is not used for the generation of the data processing sequence, in addition to distinguishing the node and the parameter, and
the type table generating process generates the type table where the decoding type for distinguishing the node represented by the gene, the parameter, and the unused gene is associated with each of the genes forming the individual from which the data processing sequence is generated.

5. The method according to claim 3, wherein
the next generation generating process includes a reference individual generating process of determining at least one of a crossover point and the gene to perform the mutation and performing at least one of the crossover and the mutation with reference to the type table.

6. The method according to claim 5, wherein
the reference individual generating process includes a type-referenced mutation process of performing the mutation of the gene associated with the parameter as the decoding type, of the plurality of genes of the individual that is a subject of the mutation, when generating the individual by the mutation.

7. The method according to claim 6, wherein
the next generation generating process further includes a normal individual generating process of performing at least one of crossover and mutation without referring to the type table, and thus generating the individual, and
the next generation generating process executes at least one of the reference individual generating process and the normal individual generating process, based on a predetermined generation condition.

8. The method according to claim 7, wherein
the generation condition is a condition that the type-referenced mutation process is executed for the individual from which the data processing sequence with the degree of similarity at up to an N-th place from the top, N being an integer equal to or greater than 1, is generated, of the individuals whose degree of similarity is high among the plurality of individuals generated by the previous routine.

9. A device for generating a data processing sequence,
the data processing sequence having an input layer, a filter layer, and an output layer, as nodes,
the device comprising:
a population generating unit that generates a plurality of individuals in which a plurality of genes for expressing one of a type of the input layer and the filter layer, and a parameter set for the filter layer, are sequenced, and thus generates a population of one generation;
a gene translation unit that allocates one of the input layer, which is one of the nodes, the filter layer, which is another one of the nodes, and the parameter, to each of the genes forming the individual, with reference to a reference table establishing a correspondence between the gene, and the node and the parameter, and thus generates the data processing sequence corresponding to the individual;
a calculation unit that performs data processing of subject data for each of a plurality of the data processing sequences expressed by the population, then compares output data outputted for each of the data processing sequences, and target data to be a target associated with the subject data, and calculates an evaluation value indicating a degree of similarity between the output data and the target data; and
a specifying unit that specifies the data processing sequence that satisfies a predetermined criterion, using a plurality of the evaluation values corresponding to the plurality of the data processing sequences, respectively.

10. A non-transitory computer-readable storage medium storing a computer program for causing a computer to execute generation of a data processing sequence,
the data processing sequence having an input layer, a filter layer, and an output layer, as nodes,
the computer program comprising:
(a) a function of generating a plurality of individuals in which a plurality of genes for expressing one of a type of the input layer and the filter layer, and a parameter set for the filter layer, are sequenced, and thus generating a population of one generation;
(b) a function of allocating one of the input layer, which is one of the nodes, the filter layer, which is another one of the nodes, and the parameter, to each of the genes forming the individual, with reference to a reference table establishing a correspondence between the gene, and the node and the parameter, and thus generating the data processing sequence corresponding to the individual;
(c) a function of performing data processing of subject data for each of a plurality of the data processing sequences expressed by the population, then comparing output data outputted for each of the data processing sequences, and target data to be a target associated with the subject data, and calculating an evaluation value indicating a degree of similarity between the output data and the target data; and
(d) a function of specifying the data processing sequence that satisfies a predetermined criterion, using a plurality of the evaluation values corresponding to the plurality of the data processing sequences, respectively.
